(19) United States
(12) Patent Application Publication
(10) Pub. No.: US 2026/0184749 A1
(43) Pub. Date: Jul. 2, 2026

(54) CARBOHYDRATE BINDING POLYPEPTIDE OF SAVALIA SAVAGLIA

(71) Applicants: University of Belgrade, Belgrade (RS); University of Ljubljana, Ljubljana (SL); Jozef Stefan Institute, Ljubljana (SL)

(72) Inventors: Uros Andjelkovic, Belgrade (RS); Ivana Vukasinovic, Belgrade (RS); Dusan Sladic, Belgrade (RS); Jurij Lah, Ljubljana (SL); Marko Fonovic, Ljubljana (SL)

(73) Assignees: University of Belgrade, Belgrade (RS); University of Ljubljana, Ljubljana (SL); Jozef Stefan Institute, Ljubljana (SL)

(21) Appl. No.: 18/696,725

(22) PCT Filed: Oct. 1, 2021
(86) PCT No.: PCT/RS2021/000013

§ 371 Date: Mar. 28, 2024

(51) Int. Cl. C07K 14/435; A61K 38/00; A61P 31/04; A61P 31/10

(52) U.S. Cl. CPC C07K 14/43504; A61P 31/04; A61P 31/10; A61K 38/00

(57) ABSTRACT

The present invention relates to a novel polypeptide which displays specific carbohydrate binding activity, particularly against glycans that contain mannose, and in vivo and ex vivo methods of use thereof. Methods of use comprise administering a polypeptide of the invention, for example to a sample or a subject in which carbohydrate is present, under conditions suitable for the polypeptide to bind the carbohydrate. The carbohydrate may be expressed by a pathogen. The invention relates to polypeptides and compositions thereof with anti-pathogen activity, such as anti-viral, anti-bacterial, anti-fungal or anti-tumour activity. Also provided are methods for the prevention or treatment of diseases and conditions mediated by pathogens which express carbohydrate, for example as glycoproteins.

### SEQUENCE LISTING

The entire contents of a Sequence Listing now titled “N429350US_REVISED.txt” created on Sep. 26, 2025 and having a size of 12,862 bytes, which has been submitted in electronic form in connection with the present application, is hereby incorporated by reference herein in its entirety.

### FIELD OF THE INVENTION

The present invention relates to a novel polypeptide which displays specific carbohydrate binding activity, particularly against glycans that contain mannose, and in vivo and ex vivo methods of use thereof. Methods of use comprise administering a polypeptide of the invention, for example to a sample or a subject in which carbohydrate is present, under conditions suitable for the polypeptide to bind the carbohydrate. The carbohydrate may be expressed by a pathogen. The invention relates to polypeptides and compositions thereof with anti-pathogen activity, such as anti-viral, anti-bacterial, anti-fungal or anti-tumour activity. Also provided are methods for the prevention or treatment of diseases and conditions mediated by pathogens which express carbohydrate, for example as glycoproteins.

### BACKGROUND OF THE INVENTION

Carbohydrate-binding proteins, other than immunoglobulins and receptors of free mono- or disaccharides for transport or chemotaxis, that display no enzymatic activity towards the recognized sugars (carbohydrates/glycans) are known as lectins. Lectins are found in virtually all organisms: prokaryotes, sea corals, algae, fungi, higher plants, invertebrates, vertebrates. Carbohydrate recognition occurs in a large number of different biological contexts. Therefore, the lectins are a highly diverse group of proteins consisting of many unrelated protein families. Different lectin families are in general structurally unrelated (BBA 1572 198). Carbohydrate specificities of lectins are different. Even those lectins that can be classified, for example, in the group of mannose binding lectins when analyzed by glycan array don not exhibit completely identical carbohydrate specificities (Consortium for Functional Glycomics). Recognition between proteins and glycans is the major mechanism for transfer of biological information that is codded in glycans (so called “the sugar code”). This information is essential in many biological processes including, intra- and intercellular transport processes, sensor branches of innate immunity, regulation of cell-cell (matrix) adhesion or migration and positive/negative growth control with implications for differentiation and malignancy (BBA 1572 165).

Lectins have numerous useful applications in medicine, biotechnology and life science. Different lectins exhibit antibacterial, antifungal, antiviral or antitumor activities. Especially strong antiviral activity has been observed for different plant and animal lectins. A well-defined class of anti-HIV agents that act as viral entry inhibitors are mannose binding lectins. Many mannose specific lectins (Hippeastrum hybrid agglutinin, Galanthus nivalis agglutinin, actinohivin, Microcystis viridis lectin, banana lectin, Oscillatoria agardhii agglutinin, Cymbidium hybrid lectin, Epipactis helleborine lectin, Listera ovate lectin, etc.) were considered as potential anti-HIV agents (AR 18 191, AR 71 237, JBC 280 41005, COV 0 95). However, cytotoxicity, agglutination of human erythrocytes, mitogenicity (mitogenic stimulation of peripheral blood mononuclear cells), inflammatory activity or low genetic barrier against mutated HIV strains are obstacles for medical application of majority of natural lectins (AR 71 237, JV 80 8411).

Nonetheless, two lectins cyanovirin-N (AMB 86 805) and scytovirin are active components of already commercialized drugs, while griffithsin is in the third phase of clinical trials. These lectins inhibit viral entry into the cells but also inhibit cell-cell viral transfer and inhibit the syncytium formation between HIV-infected and noninfected CD4+ T lymphocytes (JAC 69 582). The mechanism of action includes prevention of interaction between HIV envelope glycoprotein (GP120) and the cellular receptor protein (CD4) and a co-receptor (mainly CXCR4 or CCR5) (JBC 280 41005). Differences in carbohydrate specificity between mannose specific lectins has been exploited for improvement of anti-HIV activity, since combinations of different lectins can show synergistic activity against HIV i.e. improvement of EC50 values (ARHR 28 1513). Moreover, exposure of HIV-1 to a combination of two carbohydrate-binding agents markedly delays drug resistance development and selects for virus strains with compromised fitness (JAC 69 582). In addition, viral mutations to escape lectin drugs, such as deletion of highly conserved N-glycosylation sites, can result in loss of at least part of the viral glycan shield and consequently the exposure of previously hidden immunogenic epitopes on GP120. This may allow efficient immunological suppression of virus replication and virus clearance from the systemic circulation. In other words, when HIV mutates to circumvent effects of lectin it might become more susceptible to the immune system of the host.

HIV is not the only pathogen or even the only virus which expresses glycoproteins that could be targeted by lectins. Enveloped viruses, human and animal, that can be neutralized with different mannose specific lectins include Hepatitis C (HCV) and B virus (HBV), Influenza A and B virus, SARS-coronaviruses (SARS-COV-1 and SARS-Cov-2), Ebola virus, Dengue virus, Herpes simplex virus 1 (HSV-1) and 2 (HSV-2), West Nile virus, Yellow fever virus, Human cytomegalovirus (HCMV), Japanese encephalitis virus, Porcine epidemic diarrhea virus, Simian immunodeficiency virus (SIV) (MP 74 330).

Given the multiple applications for lectins outlined above, and the particular need for more and better antiviral drugs in light of the COVID-19 pandemic, there is a clear need for additional agents of this type, which exhibit antiviral activity without the problems associated with some known lectins. There is a general need for antiviral agents which can treat or prevent a viral infection or a disease or complication associated with a viral infection.

### SUMMARY OF THE INVENTION

The present inventors have identified, purified and characterised a novel lectin from Savalia (Gerardia) savaglia. The inventors have identified four very closely-related isoforms of this lectin (see Table 1). The sequences of the isoforms are provided as SEQ ID NOs: 1, 2, 3 and 4. SEQ ID NO: 1 is the sequence of the most prevalent (dominant) form expressed in Savalia (Gerardia) savaglia (as determined by mass spectrometry). All four isoforms of the lectin have comparable properties and they may be used interchangeably or as a mixture. The lectin may be referred to herein as Sava lectin or Savalithsin. This term may be used to refer to a mixture comprising the four isoforms, which may optionally include other isoforms or variants as defined herein. References to recombinantly produced Savalithsin typically refer to the dominant form.

Each of SEQ ID NOs: 1, 2, 3 and 4 has less than 40% sequence identity to other known lectins. Sava lectin exhibits specific binding to different glycan structures as compared to known lectins. Sava lectin is able to bind to laboratory strains and isolates of exemplary human viruses, typically enveloped human viruses including HIV, HSV2, Sars-COV-2 and Influenza A. Anti-viral activity (inhibition of infection, and/or inhibition of transmission and/or growth) has been demonstrated. Sava lectin exhibits inhibition of growth of some bacteria and yeast species, and the ability to bind to cell walls of some yeasts. Sava lectin has high stability in alkaline environment up to pH 10, and thermal stability up to 75° C., permitting storage in a range of conditions. Unlike most known plant and animal lectins, Sava lectin exhibits no significant mitogenic effect on human peripheral blood mononuclear cells. Even high doses of Sava lectin are not lethal against healthy human cells. Taken together, this suggests that Sava lectin may be a particularly useful for multiple applications, including as a therapeutic or prophylactic agent administered to patients and/or used as a topical microbicide.

### Provided Herein is:

A polypeptide, optionally an engineered polypeptide, having specific carbohydrate-binding activity, wherein said polypeptide comprises, consists essentially of, or consists of:
(a) the amino acid sequence of any one of SEQ ID NOs: 1, 2, 3 or 4;
(b) an amino acid sequence which is a fragment of the sequence of any one of SEQ ID NOs: 1, 2, 3 or 4, which fragment comprises at least 8 contiguous amino acids of the said sequence;
(c) an amino acid sequence which is at least 50% identical to the amino acid sequence of any one of (a), (b) or (c);

optionally wherein said polypeptide is engineered to include an additional methionine at the N terminus and/or a protein purification or other tag at the N and/or C terminus, which tag may be joined to the terminus by a linker.

The polypeptide may be provided in a composition, typically comprising an effective amount of a preservative. The composition may be suitable for use as a surface microbicide. The composition may be suitable for administration to a subject. The polypeptide may be immobilised.

The polypeptides of the invention are useful in various methods for binding carbohydrates. Thus, provided herein are methods comprising administering a polypeptide of the invention, for example to a sample or a subject in which carbohydrate is present. The carbohydrate may be present as a glycan component expressed on the surface of a pathogen. The pathogen may be a virus, a bacterium, a fungus, or a cancer cell. The polypeptide may thus have antibacterial, antifungal, antiviral or antitumor activity. The method may be a method treating or preventing an infection of the said pathogen, or a disease or complication associated with said infection, the method comprising administering a polypeptide of the invention to a subject.

The present invention provides a nucleic acid encoding a polypeptide of the invention. The invention also provides a host cell, such as a bacterial or plant cell, comprising such a nucleic acid or vector.

### BRIEF DESCRIPTION OF THE FIGURES

FIG. 1. Left: Flow diagram of isolation and purification procedure; Right: SDS-PAGE of Sava lectin isolate (line 1-MW makers, line 2 and 3-Sava lectin 0.1 μg/μl and 1 μg/μl respectively)

FIG. 2. Molecular weight determination of Sava lectin. Top: MLADI-TOF mass spectra; Bottom: Size exclusion chromatography at Superdex 75 chromatographic column shows Ve of 12.7 ml for Sava lectin that correspond to molecular weight of ˜16.9 kDa (Inset show elution volumes (Ve) of proteins used for molecular weight calibration).

FIG. 3. Flow diagram of primary structure elucidation

FIG. 4. Thermodynamic parameters of mannose and methyl mannoside binding to Sava lectin

FIG. 5. Differential scanning calorimetry thermogram of Sava lectin, alone and in the presence of carbohydrates.

FIG. 6. Spectrofluorimetric analysis of Sava lectin denaturation by GdmCl.

Top: denaturation curves at different temperatures; Middle: fluorescence of 1-anilino-8-naphthalene sulfonate (ANS) in the mixture with Sava lectin at different concentrations of GdmCl; Bottom: fluorescence intensity of Sava lectin, alone and in the presence of mannose, in different solutions of GdmCl.

FIG. 7. Circular dichroism (CD) spectra of Sava lectin (0.6 mg/mL), alone and in the presence of carbohydrates. Top: Far-UV CD spectra; Bottom: Near-UV CD spectra.

FIG. 8. Tertiary structure of Sava lectin determined with X-ray crystallography. Top-left: crystal of Sava lectin and X-ray diffraction pattern; Top-middle: ribbon diagram of Sava lectin structure; Top-right: ribbon diagram of computer proposed Sava lectin dimer; Lower: space-filling model of Sava lectin from different views.

FIG. 9. Computational study of Sava lectin structure. Top: ribbon structure with binding carbohydrate sites and amino acids responsible for carbohydrate binding; Bottom: model of Sava lectin multimerisation.

FIG. 10. Oligomerization of Sava lectin. Top: MLADI-TOF mass spectra of Sava lectin; Bottom: SDS-PAGE of Sava lectin samples shortly exposed to cross linker glutaraldehyde (line 1-MW markers, line 2-Sava lectin 10 μg/μl, line 3-Sava lectin 5 μg/μl and line 4-Sava lectin 1 μg/μl)

FIG. 11. Effect of Sava lectin on metabolic activity of PBMCs (peripheral blood mononuclear cells). Top: flow-diagram of experiment.

FIG. 12. Effect of Sava lectin on metabolic activity of TZM-bl cell line.

Top: flow-diagram of experiment.

FIG. 13. Effect of Sava lectin on viability of CaCo-2 cell line. (immortalized cell line of human colorectal adenocarcinoma cells). Top: 25,000 cells per well; Middle: 10,000 cells per well. The sets of marks on the left and on the right in each chart represent two different batches of Sava lectin. Each batch was assayed in triplicate. Bottom: flow-diagram of experiment.

FIG. 14. Effect of Sava lectin on viability of A549 cell line (adenocarcinome human alveolar basal epithelial cells). The sets of marks on the left and on the right in the chart represent two different batches of Sava lectin. Each batch was assayed in triplicate. Top: flow-diagram of experiment.

FIG. 15. Effect of Sava lectin on viability of ELVIS cell line. The left and right panels represent two different batches of Sava lectin. Each batch was assayed in triplicate. Top: flow-diagram of experiment.

FIG. 16. Effect of Sava lectin on viability of VeroE6 cell line. The sets of marks on the left and on the right in the chart represent two different batches of Sava lectin. Each batch was assayed in triplicate. Top: flow-diagram of experiment.

FIG. 17. Sava lectin inhibition of TZM-bl cell infection by different strains of HIV-1

Top: Flow diagram of experiment. Molarity was calculated taking into account molecular weight of Sava lectin monomer.

FIG. 18. Sava lectin inhibition of PBMCs infection by two different strains of HIV-1, Left: NL4-3; Right: NL4-3 92TH14. Top: Flow diagram of experiment. Molarity was calculated taking into account molecular weight of Sava lectin monomer.

FIG. 19. Sava lectin inhibition of ELVIS cell infection by Herpes simplex virus. Top: Flow diagram of experiment. Left: HSV-1; Right: HSV-2. The sets of marks on the left and on the right in each chart represent two different batches of Sava lectin.

FIG. 20. Sava lectin inhibition of Vero cell infection by HSV-2. Top: Flow diagram of experiment. Molarity was calculated taking into account molecular weight of Sava lectin monomer.

FIG. 21. Sava lectin inhibition of CaCo-2 cell infection by SARS-COV-2. Top: Flow diagram of experiment. Left: Pseudotype; Right: Wild-type. The sets of marks on the left and on the right in each chart represent two different batches of Sava lectin.

FIG. 22. Confocal microscopy picture of fluorescein isothiocyanate (FITC) labeled Sava lectin in interaction with Saccharomyces cerevisiae cell wall glycans.

FIG. 23. Western blot detection of glycoproteins by Sava lectin. Biotin labeled Sava lectin was visualized by Streptavidin-HRP (horseradish peroxidase) and DAB (3,3′-diaminobenzidine). Line 1 and 2: Invertase (S. cerevisiae external glycoprotein that contains mannose type N-glycans) 1 μg and 0.1 μg respectively; Line 3: extract of plasma membrane proteins from HeLa cells, 10 μg of total proteins.

FIG. 24. Expression of Sava lectin. Top: success of expression in Nicotiana benthamiana was confirmed by co-expression of Green fluorescent protein-bright patches visible on leaves; Bottom: SDS-PAGE of different time points of E. coli expression, black frame shows Sava lectin.

### BRIEF DESCRIPTION OF THE SEQUENCES

SEQ ID NO: 1 is the full length amino acid sequence of a 16,803 Da variant of Sava lectin. It contains 149 amino acids. It is the dominant form.

SEQ ID NO: 2 is the full length amino acid sequence of a 16,769 Da variant of Sava lectin. It contains 149 amino acids. There is one substitution relative to SEQ ID NO: 1, namely Phe61Leu.

SEQ ID NO: 3 is the full length amino acid sequence of a 16,617 Da variant of Sava lectin. It contains 148 amino acids. It is identical to SEQ ID NO: 1 except for the deletion of C terminal Trp.

SEQ ID NO: 4 is the full length amino acid sequence of a 16,583 Da variant of Sava lectin. It contains 148 amino acids. It is identical to SEQ ID NO: 2 except for the deletion of C terminal Trp (i.e. it includes the Phe61Leu relative to SEQ ID NO: 1).

SEQ ID NO: 5 is an exemplary nucleotide sequence encoding a polypeptide with the sequence of SEQ ID NO: 1. For expression in bacterial cells, e.g. E coli, the sequence may be modified to include an additional 5′ codon for an extra N terminal methionine. The sequence may typically be inserted into a vector for expression in bacterial cells. For example, the sequence may be inserted into vector pET28a+ using NcoI/XhoI restriction sites.

SEQ ID NOs: 6, 7, 8 and 9 correspond to SEQ ID NOs: 1 to 4, respectively, each with an additional N terminal methionine.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting. All publications, patents and patent applications cited herein, whether supra or infra, are hereby incorporated by reference in their entirety.

As used in this specification and the appended claims, the singular forms “a”, “an”, and “the” include plural referents unless the content clearly dictates otherwise. Thus, for example, reference to “a polypeptide” includes “polypeptides”, and the like.

### Polypeptides Having Specific Carbohydrate Binding Activity

### Functional Features of a Polypeptide Having Specific Carbohydrate Binding Activity

This section sets out the functional features of a polypeptide having carbohydrate binding activity, which apply in addition to the structural features outlined in the immediately following section.

The polypeptide preferably exhibits one or more of the following characteristics:
Specific carbohydrate binding activity, preferably to glycans that contain mannose (e.g. to mannose and/or methyl mannoside); and/or
Binding to and/or anti-pathogen activity against a pathogen which expresses a carbohydrate specifically bound by the polypeptide.

The ability of a polypeptide to bind specifically to a carbohydrate may be assessed by any suitable method. One such method involves immobilising a test polypeptide, e.g. on sepharose in a spin column, followed by incubation with a sample containing the carbohydrate. If the test polypeptide has carbohydrate binding ability, the carbohydrates will be detectable bound to the column or in a subsequent eluent. Alternative techniques include isothermal titration calorimetry (ITC), turbidimetric activity assay, microscale thermophoresis (MST) experiments, differential scanning calorimetry, surface plasmon resonance, glycan micro array or ELISA (e.g. competitive ELISA). Exemplary assays are described in the examples. If a measure of binding specificity for different targets is required, the binding to the different targets must be assessed using the same assay.

For the purposes of this disclosure, a polypeptide is said to exhibit “specific binding” if it reacts or associates more frequently, more rapidly, with greater duration and/or with greater affinity with a particular target than it does with alternative targets. It is also understood that, for example, a polypeptide that specifically binds to a first carbohydrate target may or may not specifically or preferentially bind to a second carbohydrate target. As such, “specific binding” or “preferential binding” does not necessarily require (although it can include) exclusive binding. Generally, but not necessarily, reference to binding means preferential binding.

The polypeptide of the invention may exhibit specific binding to glycans that contain mannose (e.g. to mannose and/or methyl mannoside). It may exhibit preferential binding for mannose and/or methyl mannoside as compared to galactose, N-acetyl galactosamine, glucose, N-acetyl glucosamine, fucose, mannitol, rhamnose, lactose, raffinose, and arabinose, when assessed using the same assay. The polypeptide may exhibit high affinity binding to mannose and/or methyl mannoside, and may exhibit very low or no detectable binding to galactose, N-acetyl galactosamine, glucose, N-acetyl glucosamine, fucose, mannitol, rhamnose, lactose, raffinose, and arabinose, when assessed using the same assay. The polypeptide may exhibit binding to D-mannose with a binding constant (Kb) of about 2500 M⁻¹ or higher, such as about 2514 M⁻¹; and/or binding to methyl alpha-D-mannoside with a Kb of about 1200 M⁻¹ or higher, such as about 1270 M⁻¹, typically when determined by isothermal titration calorimetry (ITC). There is preferably no detectable binding to galactose, N-acetyl galactosamine, glucose, N-acetyl glucosamine, fucose, mannitol, rhamnose, lactose, raffinose, and arabinose when assessed using ITC.

The carbohydrate specifically bound by the polypeptide may be present as a glycan expressed on the surface of the pathogen, for example incorporated as a component of a glycoprotein. The polypeptide may bind to the said pathogen. The polypeptide preferably exhibits strong binding to the pathogen. The polypeptide may also exhibit anti-pathogen activity ex vivo. Anti-pathogen activity may be assessed ex vivo by any suitable method for a given type of pathogen. Exemplary methods are recited in the Examples for different types of pathogen. The pathogen may be a virus, a bacterial cell, a fungal cell, or a cancer cell. Anti-pathogen activity may typically refer to inhibition of growth of the pathogen. Where the pathogen is a cell, anti-pathogen activity typically refers to inhibition of cell replication. Where the pathogen is a virus, anti-pathogen activity typically refers to inhibition of virus-induced decrease of cell viability.

The virus typically expresses at its surface at least one glycoprotein or other structure incorporating a glycan that is specifically bound by the polypeptide. The virus may express high-mannose glycan structures at its surface. The virus may be an enveloped virus, in which case the glycan is typically present in the envelope. The virus may be Human Immunodeficiency Virus (HIV), Herpex Simplex Virus 2 (HSV-2), Herpex Simplex Virus 1 (HSV-1), Sars-COV-2, Sars-COV-1, Influenza A, Influenza B, Hepatitis C (HCV) and B virus (HBV), Ebola virus, Dengue virus West Nile virus, Yellow fever virus, Human cytomegalovirus (HCMV), Japanese encephalitis virus, Porcine epidemic diarrhea virus, Simian immunodeficiency virus (SIV). The virus is preferably Human Immunodeficiency Virus (HIV), Herpex Simplex Virus 2 (HSV-2), Influenza A, or a Sars-coronavirus, such as Sars-COV-2.

The bacterial cell typically expresses at the cell surface at least one glycoprotein or other structure incorporating a glycan that is specifically bound by the polypeptide. The bacterium may express high-mannose glycan structures at its surface. The bacterium may be of the species: S. aureus, P. aeruginosa, E. coli, B. cereus, E. faecalis or S. typhimurium, and is preferably preferably S. aureus or E. coli.

The fungal cell may be a yeast cell, which typically expresses at the cell surface at least one glycoprotein or other structure incorporating a glycan that is specifically bound by the polypeptide. The fungus may express high-mannose glycan structures at its surface. The fungus may be a yeast of the species S. cerevisiae or C. albicans;

The cancer cell typically expresses at the cell surface at least one glycoprotein or other structure incorporating a glycan that is specifically bound by the polypeptide. The cancer preferably expresses said glycan at a higher level than healthy cells. The cancer cell may express high-mannose glycan structures at its surface. The cancer cell may be human. It may be a lung cancer cell (e.g. alveolar basal epithelial cell) or a colorectal cancer cell (e.g. intestinal epithelial cell). It is preferably an adenocarcinoma cell. The cancer cell may alternatively be from a myeloid or lymphocytic leukemia.

The polypeptide preferably has low or no mitogenic effects on healthy human cells, such as peripheral blood mononuclear cells (PBMCs). Mitogenic effects may be assessed by any suitable method. Exemplary methods are recited in the Examples.

The polypeptide preferably has low or no cytotoxicity for healthy human cells, such as peripheral blood mononuclear cells (PBMCs). Cytotoxicity may be assessed by any suitable method. Exemplary methods are recited in the Examples.

The carbohydrate binding activity of the polypeptide is preferably maintained over a wide pH range, such as from pH 4.0 to 10.0, from pH 5.0 to 9.0, or from pH 6.5 to pH 9.0, optionally with maximum activity over the range of from pH 6.5 to 7.5, preferably with maximum activity at about pH 7.0. Stability of carbohydrate binding activity at different pHs may be assessed by any suitable method. Exemplary methods are recited in the Examples.

The carbohydrate binding activity of the polypeptide is preferably maintained over a wide temperature range, such as from about 0° C. to about 75° C. A preferred range for activity is about 4° C. to about 45° C. Optimal activity is typically at 37° C. Stability of carbohydrate binding activity at different temperatures may be assessed by any suitable method. Exemplary methods are recited in the Examples.

### Structural Features of a Polypeptide Having Specific Carbohydrate Binding Activity

This section sets out the structural features of a polypeptide having specific carbohydrate binding activity, which apply in addition to the functional features outlined in the immediately preceding section.

The polypeptide is typically no longer than about 200 amino acids in length. The polypeptide may comprise the 148 or 149 amino acids of SEQ ID NO: 1, 2, 3 or 4, and in addition may be engineered to include one or more additional amino acids up to a total length of 200 amino acids, wherein said additional amino acids are typically to assist with production, isolation or purification. The polypeptide may thus comprise, comprise, consist essentially, or consist of the sequence of any one of SEQ ID NOs: 1, 2, 3 or 4. The polypeptide may have a maximum length of 190, 180, 170, 160, or 150 amino acids.

The polypeptide preferably comprises, consists essentially of, or consists of any shorter fragment of contiguous amino acids from SEQ ID NO: 1, 2, 3, or 4, provided it has specific carbohydrate binding activity. The polypeptide may comprise, consist essentially of, or consist of 8, 9, 10, 11, 12, 13, 14, 15, 16, 17, 18, 19, 20, 30, 40, 50, 60, 70, 80, 90, 100, 110, 120, 130, 140, 141, 142, 143, 144, 145, 146, 147 or 148 contiguous amino acids of any one of SEQ ID NOs: 1, 2, 3 or 4. provided that the polypeptide has specific carbohydrate binding activity. The fragment preferably binds to the mannose with an affinity that is at least 50% of the affinity for mannose of a mixture of the four variants of Sava lectin (i.e. a mixture of the polypeptides of SEQ ID NOs: 1, 2, 3, and 4), or of the polypeptide consisting of the sequence of SEQ ID NO: 6, when measured in the same assay. The fragment more preferably binds to the mannose with an affinity that is comparable to or superior to the affinity for mannose of a mixture of the four variants of Sava lectin (i.e. a mixture of the polypeptides of SEQ ID NOs: 1, 2, 3, and 4), or of the polypeptide consisting of the sequence of SEQ ID NO: 6, when measured in the same assay.

The sequence of SEQ ID NO: 1, 2, 3 or 4, or any fragment of SEQ ID NO: 1, 2, 3, or 4 may be engineered to include one or more additional amino acids, wherein said additional amino acids are typically to assist with production, isolation or purification. For example, the sequence may be engineered to include an additional N terminal methionine. SEQ ID NOs: 6, 7, 8 and 9 correspond to SEQ ID NOs: 1 to 4, respectively, each with an additional N terminal methionine.

Alternatively, the polypeptide of the invention may comprise, consist essentially, or consist of a variant of the amino acid sequence of any one of SEQ ID NOs: 1, 2, 3 or 4, or any fragment thereof as defined above, provided the variant has specific carbohydrate binding activity. Said variant may be at least 50% identical to the amino acid sequence of any one of SEQ ID NOs: 1, 2, 3 or 4, or any fragment thereof as defined above. The variant sequence may be at least 60%, at least 70%, at least 80%, at least, 85%, at least 90%, at least 95%, at least 98% or at least 99% identical to the sequence of any one of SEQ ID NOs: 1, 2, 3 or 4, or any fragment thereof as defined above. The identity level is preferably at least 85% or higher. Identity relative to the sequence of any one of SEQ ID NOs: 1, 2, 3 or 4, or any fragment thereof as defined above can be measured over a region of at least 30, 40, 50, 60, 70, 80, 90, 100, 110, 120, 130, 140, 141, 142, 143, 144, 145, 146, 147 or 148 or more contiguous amino acids of the sequence shown in any one of SEQ ID NOs: 1, 2, 3 or 4, or any fragment thereof as defined above. More preferably, identity is measured over the full length of any one of SEQ ID NOs: 1, 2, 3 or 4, or any fragment thereof as defined above. Identity to SEQ ID NOs: 1, 2, 3 or 4, or any fragment thereof may be measured over the length of the variant, provided the variant is of a length which is no more than 50 amino acids longer or shorter than the reference sequence, and is preferably of approximately (or exactly) the same length as the reference sequence. SEQ SEQ ID NOs: 1, 2, 3 or 4 are the most preferred reference sequences. Identity of a variant is most preferably measured over the full length of any one of SEQ ID NOs: 1, 2, 3 or 4.

Amino acid identity may be calculated using any suitable algorithm. For example the PILEUP and BLAST algorithms can be used to calculate identity or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al (1984) Nucleic Acids Research 12, 387-395).

The sequence of a polypeptide of the invention may comprise a variant of the amino acid sequence of any one of SEQ ID NOs: 1, 2, 3 or 4, or any fragment thereof as defined above, having specific carbohydrate binding activity, in which modifications, such as amino acid additions, deletions or substitutions are made relative to the sequence of SEQ ID NOs: 1, 2, 3 or 4, or said fragment. Additional amino acids may be included e.g. to assist with production, isolation or purification. For example, the sequence of a variant may be engineered to include an additional N terminal methionine. Amino acid substitutions may also be introduced to improve protein characteristics such as temperature stability, pH stability, stability in general, to improve carbohydrate binding, or to reduce binding to unwanted binding partners (such as those that induce cell toxicity).

Unless otherwise specified, the modifications are preferably conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A1 below. Where amino acids have similar polarity, this can be determined by reference to the hydropathy scale for amino acid side chains in Table A2. A sequence of a polypeptide of the invention may comprise a variant of the amino acid sequence of any one of SEQ ID NOs: 1, 2, 3 or 4 in which up to 5, 10, 15, 20, 25, 30, 35, 40, 45, 50, 55, 60, 65 or 70 conservative substitutions are made.

TABLE A1
Chemical properties of amino acids

| | | | |
|---|---|---|---|
| Ala (A) | aliphatic, hydrophobic, neutral | Met (M) | hydrophobic, neutral |
| Cys (C) | polar, hydrophobic, neutral | Asn (N) | polar, hydrophilic, neutral |
| Asp (D) | polar, hydrophilic, charged (−) | Pro (P) | hydrophobic, neutral |
| Glu (E) | polar, hydrophilic, charged (−) | Gln (Q) | polar, hydrophilic, neutral |
| Phe (F) | aromatic, hydrophobic, neutral | Arg (R) | polar, hydrophilic, charged (+) |
| Gly (G) | aliphatic, neutral | Ser (S) | polar, hydrophilic, neutral |
| His (H) | aromatic, polar, hydrophilic, charged (+) | Thr (T) | polar, hydrophilic, neutral |
| Ile (I) | aliphatic, hydrophobic, neutral | Val (V) | aliphatic, hydrophobic, neutral |
| Lys (K) | polar, hydrophilic, charged(+) | Trp (W) | aromatic, hydrophobic, neutral |
| Leu (L) | aliphatic, hydrophobic, neutral | Tyr (Y) | aromatic, polar, hydrophobic |

TABLE A2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A suitable modification may replace at least one amino acid in the starting sequence with an amino acid having comparable characteristics but which is not one of the 20 L-configuration amino acids appearing in natural eukaryotic proteins. For example, at least one L-configuration amino acid may be replaced with the directly corresponding non-L configuration amino acid, such as a D-configuration amino acid, or with a non-L configuration amino acid which is otherwise a conservative substitution as defined above.

The amino acid sequence of a polypeptide of the invention may comprises a variant of the amino acid sequence as described above. However, certain residues are preferably retained within the said variant sequence. For example, the discussion in the Examples of “Carbohydrate specificity” and “Thermodynamics of carbohydrate binding” suggests certain binding site characteristics that are preferably maintained.

Any polypeptide of the invention which comprises any one of SEQ ID NOs: 1, 2, 3 or 4, or any fragment therefore, having specific carbohydrate binding activity as defined above, or any variant of any one of these sequences, may optionally be engineered to include an additional methionine at the N terminus and/or protein purification or other tag at the C terminus and/or the N terminus. The tag is a sequence which is not naturally expressed in Savalia (Gerardia) savaglia as a contiguous domain of the Sava lectin protein of any one of SEQ ID NOs: 1, 2, 3 or 4. A preferred protein purification tag is a histidine tag. A histidine tag preferably consists of six histidine residues. The histidine tag may be linked to the C terminus by a linker, which is typically a short sequence of amino acids, such as 3-5 amino acids. The linker typically consists predominantly of glycine and serine residues, and may preferably include the sequence GSG. For example GSG and GSGLE are suitable linkers. SEQ ID NO: 3 is an example of an engineered sequence.

In summary therefore, provided herein is:

A polypeptide, optionally an engineered polypeptide, having specific carbohydrate-binding activity, wherein said polypeptide comprises, consists essentially of, or consists of:
(a) the amino acid sequence of any one of SEQ ID NOs: 1, 2, 3 or 4;
(b) an amino acid sequence which is a fragment of the sequence of any one of SEQ ID NOs: 1, 2, 3 or 4, which fragment comprises at least 8 contiguous amino acids of the said sequence;
(c) an amino acid sequence which is at least 50% identical to the amino acid sequence of any one of (a), (b) or (c).
   optionally wherein said polypeptide is engineered to include an additional methionine at the N terminus and/or a protein purification or other tag at the C and/or N terminus, which tag may be joined to the terminus by a linker.

### General Polypeptide Features

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. The terms “protein”, “peptide” and “polypeptide” may be used interchangeably. The term “amino acid” may refer to either natural and/or unnatural or synthetic amino acids, including both D or L optical isomers, and amino acid analogs and peptidomimetics.

A polypeptide may be produced by suitable method, including recombinant or synthetic methods. For example, the polypeptide may be synthesised directly using standard techniques known in the art, such as Fmoc solid phase chemistry, Boc solid phase chemistry or by solution phase peptide synthesis. Alternatively, a polypeptide may be produced by transforming a cell, typically a bacterial cell or a plant cell, with a nucleic acid molecule or vector which encodes said polypeptide. Production of polypeptides by expression in bacterial host cells is described below and is exemplified in the Examples. The invention provides nucleic acid molecules and vectors which encode a polypeptide of the invention. The invention also provides a host cell comprising such a nucleic acid or vector. An exemplary polynucleotide molecules encoding a polypeptide disclosed herein is provided as SEQ ID NOs: 5. Any such sequence may be modified to include at the 5′ end a codon for the N terminal methionine (ATG). Any such sequence may also be modified such that, prior to the stop codon (TAA) at the 3′ end, codons for a protein purification or other tag are included. The optional inclusion of an additional methionine and a tag are discussed in more detail elsewhere in this document.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention encodes a polypeptide of the invention and may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences, for example in an expression vector. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al (1989, Molecular Cloning—a laboratory manual; Cold Spring Harbor Press). The nucleic acid molecules of the present invention may be provided in the form of an expression cassette which includes control sequences operably linked to the inserted sequence, thus allowing for expression of the polypeptide of the invention in vivo (e.g. in prokaryotic or eukaryotic expression systems). These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

The invention also includes cells that have been modified to incorporate an expression vector as described above. Such cells may be cultured or grown using routine methods to produce a polypeptide of the invention. Such cells typically include prokaryotic cells such as bacterial cells, for example E. coli, Lactobacillus or Agrobacterium tumefaciens, or plant cells, such as cells of Nicotiana benthamiana. The plant cell may be modified to express a polypeptide using, for example, a previously transformed bacterial cell of Agrobacterium tumefaciens. Suitable methods are disclosed in the Examples.

A polypeptide may be engineered or modified to assist with production, isolation or purification. For example, where a polypeptide of the invention is produced by recombinant expression in a bacterial host cell, the sequence of the polypeptide may include an additional methionine (M) residue at the N terminus to improve expression. As another example, the polypeptide of the invention may be engineered or modified by addition of protein purification tag and the N or C terminus, preferably at the C terminus. The protein purification tag is preferably a moiety which is not naturally expressed in Savalia (Gerardia) savaglia. The protein purification tag is preferably a moiety which is not present in a wildtype polypeptide chain as expressed in a Savalia species. A protein purification tag may be a ligand which is capable of binding directly and specifically to a separation means. Alternatively, the protein purification tag may be one member of a binding pair and the separation means comprises a reagent that includes the other member of the binding pair. Any suitable binding pair can be used.

Where the polypeptide is engineered or modified by addition of one member of a binding pair, the polypeptide is preferably histidine-tagged or biotin-tagged. Typically the amino acid coding sequence of the histidine tag is included at the gene level and the polypeptide is expressed recombinantly in a bacterial or plant cell. The histidine or biotin tag is typically present at either end of the polypeptide, preferably at the C-terminus. It may be joined directly to the polypeptide or joined indirectly by any suitable linker sequence, such as 3, 4 or 5 glycine residues, or a mixture of glycine and serine residues. The histidine tag typically consists of six histidine residues, although it can be longer than this, typically up to 7, 8, 9, 10 or 20 amino acids or shorter, for example 5, 4, 3, 2 or 1 amino acids.

Alternative tags useful for protein purification include Maltose-binding protein (MBP), glutathione S-transferase (GST), thioredoxin (TRX), ubiquitin (Ub), small ubiquitin related modifier (SUMO), solubility-enhancer peptide sequences (SET), and N-utilization substance (NusA). Suitable tags are also discussed in Costa et al; Front Microbiol. 2014; 5:63, which is incorporated by reference, including in particular each of the tags that are disclosed in Table 1 of Costa et al.

The amino acid sequence of a polypeptide may be modified or engineered to include at least one non-naturally occurring amino acid, for example to increase stability. When the polypeptides are produced by synthetic means, such amino acids may be introduced during production. The polypeptides may also be modified following either synthetic or recombinant production. Polypeptides may also be produced using D-amino acids. In such cases the amino acids will be linked in reverse sequence in the C to N orientation. This is conventional in the art for producing such polypeptides. Preferred polypeptides of the invention may be engineered to include at least one such unnatural or synthetic amino acid, or at least one non-L configuration amino acid.

A number of side chain modifications are known in the art and may be made to the side chains of the polypeptides, subject to the polypeptides retaining any further required activity or characteristic as may be specified herein. It will also be understood that polypeptides may be chemically modified, e.g. post-translationally modified. For example, they may be glycosylated, phosphorylated or comprise modified amino acid residues. The polypeptide may be PEGylated.

A polypeptide may be provided in a substantially isolated or purified form. That is, isolated from the majority of the other components present in a cellular extract from a cell in which the polypeptide was expressed. The polypeptide may be mixed with carriers or diluents (as discussed below) which will not interfere with the intended use and still be regarded as substantially isolated. It may also be in a substantially purified form, in which case it will generally comprise at least 90%, e.g. at least 95%, 98% or 99%, of the protein in the preparation. Where a polypeptide is provided in a composition with an additional active component, such as another polypeptide, each said polypeptide will individually be purified to a high level of homogeneity prior to mixing in an appropriate ratio for the intended purpose of each. For example, two polypeptides may be each be purified to at least 90% homogeneity prior to combining in a 1:1 ratio.

In particular embodiments, the polypeptide of the invention may be linked (directly or indirectly) to another moiety. The carbohydrate binding ability of the polypeptide may thus be used to target the other moiety to particular locations, such as the in vivo locations of the pathogens discussed above. The other moiety may be a therapeutic agent such as a drug. Targeted delivery of drugs using lectins is discussed in detail in Bies et al 2004 Advanced Drug Delivery Reviews 56; 425-435, which is herein incorporated by reference. The other moiety may be a detectable label. The other moiety may be comprise or consist of an additional protein domain having alternative or additional functions. The other moiety may be a binding moiety, such as an antibody or a polypeptide binding domain specific for a target, and accordingly the conjugated molecule may have bispecific binding activity.

The therapeutic agent or a detectable label may be directly attached, for example by chemical conjugation, to a polypeptide. Methods of conjugating agents or labels to a polypeptide are known in the art. For example, carbodiimide conjugation (Bauminger S and Wilchek M, 1980, Methods Enzymol., 70, 151-159) may be used to conjugate a variety of agents, including doxorubicin, to a polypeptide. The water-soluble carbodiimide, 1-ethyl-3-(3-dimethylaminopropyl) carbodiimide (EDC) is particularly useful for conjugating a functional moiety to a binding moiety.

Other methods for conjugating a moiety to a polypeptide can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the polypeptide maintains its carbohydrate binding ability and that the functional moiety maintains its relevant function.

The therapeutic agent linked to a polypeptide may comprise a polypeptide or a polynucleotide encoding a polypeptide which is of therapeutic benefit. Examples of such polypeptides include anti-proliferative or anti-inflammatory cytokines. The therapeutic agent may be an anti-viral, anti-bacterial, anti-fungal or anti-cancer agent. The therapeutic agent may be a cytotoxic agent.

The antibody may be linked to a detectable label. By “detectable label” it is meant that a polypeptide is linked to a moiety which, when located at the target site following administration of a polypeptide into a patient, may be detected, typically non-invasively from outside the body and the site of the target located. Thus, a polypeptide may be useful in imaging and diagnosis.

Typically, the label is or comprises a radioactive atom which is useful in imaging. Suitable radioactive atoms include 99mTc and 123I for scintigraphic studies. Other labels include, for example, spin labels for magnetic resonance imaging (MRI) such as 123I again, 131I, 111In, 19F, 13C, 15N, 17O, gadolinium, manganese or iron. Clearly, the sufficient amount of the appropriate atomic isotopes must be linked to a polypeptide in order for the molecule to be readily detectable.

The radio- or other labels may be incorporated in known ways. For example, a polypeptide may be biosynthesised or may be synthesised by chemical amino acid synthesis using suitable amino acid precursors involving, for example, fluorine-19 in place of hydrogen. Labels such as 99mTc, 123I, 186Rh, 188Rh and 111In can, for example, be attached via cysteine residues in polypeptides. Yttrium-90 can be attached via a lysine residue. Preferably, the detectable label comprises a radioactive atom, such as, for example technetium-99m or iodine-123. Alternatively, the detectable label may be selected from the group comprising: iodine-123; iodine-131; indium-111; fluorine-19; carbon-13; nitrogen-15; oxygen-17; gadolinium; manganese; iron.

The polypeptide may bind selectively to a therapeutic agent or to a detectable label, directly or indirectly. For example, the polypeptide may be linked to a moiety which selectively binds to a further compound or component which is a therapeutic agent or is readily detectable.

As a further example, a polypeptide of the invention may be conjugated to the Fc region of an antibody (preferably human IgG) in order to drive Fc-mediated effector functions including ADCC (antibody-dependent cell-mediated cytotoxicity), increased half-life in vivo, complement-dependent cytotoxicity (CDC), and antibody-dependent cell-mediated phagocytosis (ADCP) in response to a lectin-mediated carbohydrate-binding event.

A polypeptide may be provided in lyophilised form, suitable for reconstitution in aqueous solution prior to use. The lyophilised composition has improved stability enabling longer storage of the polypeptide. A polypeptide is typically substantially purified prior to freeze-drying. A method of preparing a polypeptide in lyophilised form, comprising freeze-drying said polypeptide in a suitable buffer, such as Phosphate-buffered saline (PBS), Tris-buffered saline (TBS), or another Tris-buffer is provided herein. The resulting polypeptide in lyophilised form is also provided. A method of preparing a solution of a polypeptide, comprising providing the polypeptide in lyophilised form and reconstituting with a suitable carrier or diluent, such as water, is also provided.

A polypeptide may be immobilised using methods known in the art, for example as described in Datta S et al., Enzyme immobilization: an overview on techniques and support materials, 3 Biotech, 3(1): 1-9 (2013). For example, the polypeptide may be immobilised by adsorption, covalent binding, affinity immobilization or entrapment. Materials that can be used as supports include but are not limited to for example, natural supports such as agarose, sepharose, collagen, gelatin, cellulose, pectin, sepharose, inorganic materials such as ceramics, silica, glass, activated carbon or charcoal, or synthetic polymers, such as Poly(styrene-divinylbenzene), or latex. Any of these may be provided as a resin or in any other suitable format. The polypeptide may be immobilised on magnetic beads. The polypeptide may be immobilised on a monolith support, examples of which are disclosed in Andjelković et al, (2017) Electrophoresis 38, 2851-2869 (doi: 10.1002/elps.201700260) and Kubota et al. (2017). New platform for simple and rapid protein-based affinity reactions. Scientific Reports, 7 (doi: 10.1038/s41598-017-00264-y).

The polypeptide may be immobilised on a support for any purpose, for example the polypeptide may be immobilised on a surface of a device or a component of a device:
for the detection in or removal of airborne pathogens from an environment
for the detection in or removal of pathogens from a sample, such as a sample of body fluid from a subject

### Compositions and Formulations Comprising Polypeptides

In another aspect, the present invention provides compositions comprising a polypeptide of the invention. For example, the invention provides a composition comprising one or more polypeptides of the invention, a preservative, and optionally one or more carriers, excipients, diluents or vehicles. Typically, the final composition is sterile and pyrogen free. The preservative, carrier, excipient, diluent or vehicle may preferably be pharmaceutically acceptable, in the sense of being compatible with the other ingredients of the composition and not deleterious to a subject to which the composition is administered. In this case, the composition may be referred to as a pharmaceutical composition. The composition may be suitable for microbicidal sterilization of inanimate objects, such as medical supplies or equipment, laboratory equipment and supplies, instruments, devices. The composition may be formulated for use as a topical microbicide, applied to locations typically subject to initial contact with a pathogen, for example vaginal, rectal, oral, penile tablets, creams, pessaries, tampons, creams, gels, pastes, foams, or spray formulas, anti-viral lubricants for condoms, a diaphragm, a cervical cap, a vaginal ring, and a sponge.

Formulation of a suitable composition can be carried out using standard pharmaceutical formulation chemistries and methodologies all of which are readily available to the reasonably skilled artisan. For example, the agent can be combined with a preservative, and one or more carriers, excipients or vehicles. Auxiliary substances, such as wetting or emulsifying agents, pH buffering substances, reducing agents and the like, may be present in the excipient or vehicle. Suitable reducing agents include cysteine, thioglycerol, thioreducin, glutathione and the like. Excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one embodiment of a composition for parenteral administration, the active ingredient is provided in dry (for e.g., a powder or granules) form for reconstitution with a suitable vehicle (e.g., sterile pyrogen-free water) prior to parenteral administration of the reconstituted composition. The compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parentally-administrable compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. The compositions may be suitable for administration by any suitable route including, for example, intradermal, subcutaneous, percutaneous, intramuscular, intra-arterial, intraperitoneal, intraarticular, intraosseous or other appropriate administration routes. Preferred compositions are suitable for administration by intravenous infusion.

### Methods of Use of Polypeptides

The polypeptides of the invention are useful in various methods. Thus, provided herein are methods comprising administering a polypeptide of the invention, for example to a sample or a subject. The method may be a method for the specific binding a carbohydrate, and may optionally further comprises the detection or analysis of the bound products. The method may be a method treating or preventing a disease, the method comprising administering a polypeptide of the invention to a subject.

For example, the present polypeptides may provide useful tools for biotechnology. The polypeptides may be used in a a method for the specific binding a carbohydrate. The carbohydrate may be a glycan that contain mannose (e.g. mannose and/or methyl mannoside). The polypeptide may be administered to a sample containing carbohydrate and incubated under conditions which permit binding to occur. The sample may be any body fluid taken from a subject, such as blood, plasma or sperm. The method optionally further includes determining whether or not a carbohydrate has bound and/or separating the carbohydrate and any linked glycoprotein from the resulting mixture.

The present invention may also include a method for detecting carbohydrate in a sample, wherein the method comprises contacting said sample with a polypeptide of the invention, to thereby allow formation of a polypeptide-carbohydrate complex. The said complex is also provided herein. The method may optionally include separating said polypeptide from the contacted sample and determining whether the separated polypeptide is bound to the carbohydrate, thereby determining the presence or absence of carbohydrate in the sample. The method may also be used for isolating a carbohydrate-linked glycoprotein from a sample.

In such methods, a sample is contacted with a polypeptide of the invention under conditions suitable for the polypeptide to interact with any carbohydrate in the sample and for binding to occur. Suitable conditions include incubation with a polypeptide of the invention for at least 1 minute, 2 minutes, 3 minutes, 4 minutes, 5 minutes, 6 minutes, 7 minutes, 8 minutes, 9 minutes, 10 minutes, or longer. The incubation may be for 5-10 minutes, which is typically sufficient to assess anti-viral activity. Incubation preferably takes place at room temperature, more preferably at approximately 20° C., 25° C., 30° C., 35° C., 40° C. or 45° C., and most preferably at approximately 37° C.

The methods described above may be carried out under any suitable pH. Suitable pH values include, for example, a pH of around 4.0 to 10.0. Preferred pH for the activity of a polypeptide of the invention is in the range 6.5. to 7.5. The method may be conducted in any suitable buffer, such as tris buffered saline (TBS) or phosphate buffered saline (PBS). The approximate ratio of the polypeptide of the invention to the protein content of the sample may be 1:1, 2:1, 4:1, 6:1, 10:1, 15:1, 20:1, 1:2, 1:4, or 1:6, 1:10, 1:15, 1:20, 1:40, 1:100, 1:200 or 1:400 (wt:wt). A preferred ratio is 1:1 (wt:wt).

The detection or analysis of the sample to determine whether carbohydrate has been bound may be assessed by any suitable analytical method, such as but not limited to mass spectrometry, HPLC, affinity chromatography, gel electrophoresis, SDS-PAGE, ELISA, lectin blotting, spectrometry, capillary electrophoresis and other standard laboratory techniques for analysis. For example, the molecular weight of the carbohydrate and any linked glycoprotein may be analysed.

Separation of the bound carbohydrate and any linked glycoprotein and the polypeptide of the invention may be carried out by any suitable separation means. For example, the separation means may comprise a population of magnetic nanoparticles. These may be separated from a sample using magnetic field separation, preferably high-gradient magnetic field separation. Examples of reagents or separating means are populations of magnetic particles capable of binding to the polypeptide of the invention. For example, where the polypeptide is derivatised with a histidine tag, the magnetic particles contain on their surface chelating groups which carry a nickel, copper or zinc ion. Alternatively, where the polypeptide is derivatised with a biotin tag, the magnetic particles contain on their surface streptavidin.

The separation means may also comprise a solid support to which the polypeptide of the invention is immobilised. Examples of solid supports include those described in previous sections, and may include agarose or sepharose resins, cross-linked agarose beads, or similar. The support may be used as the matrix in an affinity chromatography column. Alternatively the solid support may comprise a suitable silica-based material or polystyrene, or a plastic container such as a microtiter plate or equivalent, to which the polypeptide of the invention can be directly adsorbed.

Alternative separation means include reagents comprising antibodies specific to the polypeptide of the invention, which may be generated by methods standard in the art. Antibodies in this sense include a monoclonal antibody, a polyclonal antibody, a single chain antibody, a chimeric antibody, a CDR-grafted antibody or a humanized antibody. The antibody may be an intact immunoglobulin molecule or a fragment thereof such as a Fab, F(ab′)2 or Fv fragment. If more than one antibody is present, the antibodies preferably have different non-overlapping determinants such that they may bind to the polypeptide of the invention simultaneously. The antibody may be bound to a solid support or may be labeled or conjugated to another chemical group or molecule to assist with their separation or isolation. For example, typical chemical groups include fluorescent labels such as Fluorescein isothiocyanate (FITC) or Phycoerythrin (PE), or tags such as biotin.

Other suitable means of separation include elution of the carbohydrate and any linked glycoprotein from the (typically immobilised) polypeptide by contacting the polypeptide from the contacted sample with a suitable elution buffer. The choice of elution buffer may depend on the acid-sensitivity of the protein. Elution buffers may comprise high molar concentrations of urea (typically at least 5, 6, 7 or most preferably at least 8M) or high concentrations of a detergent (typically at least around 1%, 5% or 10%). Suitable detergents include Nonidet P40, Triton X-100, Tween 20, CHAPS, sodium deoxycholate, and RapiGest SF surfactant, but Sodium dodecyl sulphate (SDS) is preferred. High molar urea is preferred over detergent since downstream procedures are more likely to be sensitive to the presence of detergent. Particularly preferred elution buffers are glycans, such as monosaccharides. Elution of glycans or glycoproteins captured on a polypeptide of the invention may preferably be performed with monosaccharide. For example mannose may be used to elute glycoproteins captured on immobilized Sava lectin.

The sample in any of the above methods may be a sample taken from a patient, preferably a human patient. The results obtained may be used for a diagnostic purpose, for example to detect the presence of pathogens which express glycoproteins on their surface, or to detect a low abundance tumour marker. Such a use may involve comparison of the results obtained from the patient sample to those obtained using a sample obtained from a healthy control.

The polypeptides may also be used in therapy or prophylaxis. In therapeutic applications, polypeptides or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means. Thus the invention also provides a polypeptide of the invention for use in the treatment of the human or animal body. Also provided herein is a method of prevention or treatment of disease or condition in a subject, which method comprises administering a polypeptide of the invention to the subject in a prophylactically or therapeutically effective amount. The polypeptide is preferably administered by intravenous infusion, but may be administered by any suitable route including, for example, intradermal, subcutaneous, percutaneous, intramuscular, intra-arterial, intraperitoneal, intraarticular, intraosseous or other appropriate administration routes. The amount of said polypeptide that is administered may be between 0.01 mg/kg BW and 2 mg/kg BW, between 0.04 and 2 mg/kg BW, between 0.12 mg/kg BW and 2 mg/kg BW, preferably between 0.24 mg/kg and 2 mg/kg BW and most preferably between 1 mg/kg and 2 mg/kg BW.

Polypeptides of the invention may be particularly useful in a method for the treatment or prevention of a disease or condition mediated by a pathogen which expresses at its surface at least one glycoprotein or other structure incorporating a glycan that is specifically bound by the polypeptide. The invention also provides a polypeptide of the invention for use in the manufacture of a medicament for use in a method for the treatment or prevention of a disease or condition mediated by said pathogen. The pathogen may be a virus, a bacterium, a fungus, or a cancer cell. The method may be a method treating or preventing an infection of the said pathogen, or a disease or complication associated with said infection, the method comprising administering a polypeptide of the invention to a subject, typically a human subject

The method may be for the treatment or prevention of any a viral infection, or a disease or complication associated with a viral infection. The virus typically expresses at its surface at least one glycoprotein or other structure incorporating a glycan that is specifically bound by the polypeptide. The virus preferably expresses high-mannose glycan structures at its surface. The virus may be an enveloped virus, in which case the glycan is typically present in the envelope. The virus may be Human Immunodeficiency Virus (HIV), Herpex Simplex Virus 2 (HSV-2), Herpex Simplex Virus 1 (HSV-1), Sars-COV-2, Sars-COV-1, Influenza A, Influenza B, Hepatitis C (HCV) and B virus (HBV), Ebola virus, Dengue virus West Nile virus, Yellow fever virus, Human cytomegalovirus (HCMV), Japanese encephalitis virus, Porcine epidemic diarrhea virus, Simian immunodeficiency virus (SIV). The virus is preferably Human Immunodeficiency Virus (HIV), Herpex Simplex Virus 2 (HSV-2) or a Sars-coronavirus, such as Sars-COV-2. The method may be for the treatment or prevention of a disease or condition caused by any virus as defined above. The method may be for the treatment or prevention of AIDs, COVID-19 or herpes.

The method may be for the treatment or prevention of a bacterial infection, or a disease or complication associated with a bacterial infection. The bacterial cell typically expresses at the cell surface at least one glycoprotein or other structure incorporating a glycan that is specifically bound by the polypeptide. The bacterium preferably expresses high-mannose glycan structures at its surface. The bacterium may be of the species: S. aureus, P. aeruginosa, E. coli, B. cereus, E. faecalis or S. typhimurium, and is preferably preferably S. aureus or E. coli. The method may be for the treatment or prevention of a a disease or condition caused by any bacterium as defined above.

The method may be for the treatment or prevention of a fungal infection, or a disease or complication associated with a fungal infection. The fungal cell may be a yeast cell, which typically expresses at the cell surface at least one glycoprotein or other structure incorporating a glycan that is specifically bound by the polypeptide. The fungus preferably expresses high-mannose glycan structures at its surface. The fungus may be a yeast of the species S. cerevisiae or C. albicans. The method may be for the treatment or prevention of a disease or condition caused by any fungus as defined above.

The method may be for the treatment or prevention of a cancer. The cancer cell causing the cancer typically expresses at the cell surface at least one glycoprotein or other structure incorporating a glycan that is specifically bound by the polypeptide. The cancer preferably expresses said glycan at a higher level than healthy cells. The cancer cell preferably high-mannose glycan structures at its surface. The cancer cell may be human. It may be a lung cancer cell (e.g. alveolar basal epithelial cell) or a colorectal cancer cell (e.g. intestinal epithelial cell). It is preferably an adenocarcinoma cell. The cancer cell may alternatively be from a myeloid or lymphocytic leukemia. The method may be for the treatment or prevention of a cancer caused by any cancer cell as defined above.

The following Examples illustrate the invention

### Example 1

### Results

### Introduction, Basic Properties

This work presents isolation of a new antiviral protein Sava lectin from coral Savalia (Gerardi) savaglia. Previously, one antiviral protein (a mannose binding lectin) from corral Gerardia savaglia has been described (JAIDS 1 453). This protein has molecular weight of 14,800 Da, pI (isoelectric point) 4.8, molar extinction coefficient 1.27×10⁵ M⁻¹ cm⁻¹, requires Ca²⁺ for full activity and is 121 amino acids length. A primary amino acid sequence was not determined (EJB 169 97).

The antiviral protein described in this study is also a mannose binding lectin, but is demonstrably a different protein. It has different molecular weight, different pI, different molar extinction coefficient, does not require Ca²⁺ for activity, and has a different amino acid composition. The sequence of the new antiviral protein is not found in protein data banks (Uniprot or NCBI). It displays at most 40% similarity with known sequences. The average molecular weight is from 16,583 Da to 16,803 Da, see primary structure Table 1. The Sava lectin pI is 8.9

### Purification

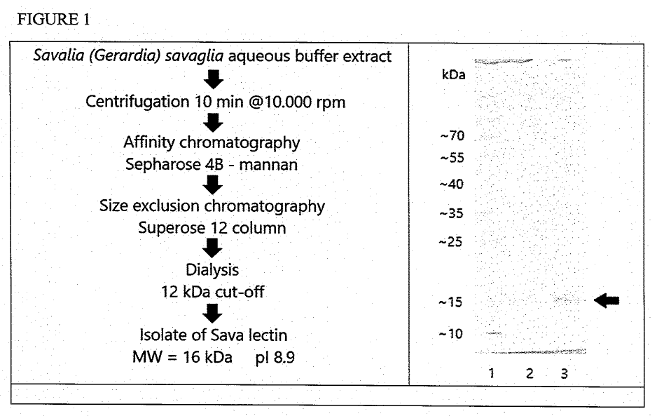

Purification of Sava lectin was performed according to flow diagram presented at FIG. 1. Purity of Sava lectin isolate was confirmed by sodium dodecyl sulphate polyacrylamide gel electrophoresis (SDS-PAGE). Single protein band around 17 kDa was obtained of high purity >95% according to densitometry, see FIG. 1.

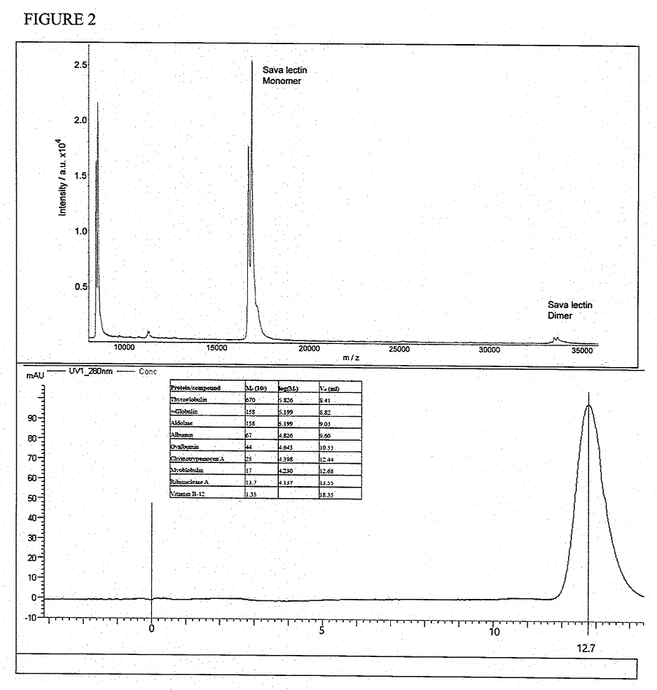

Isolated and purified preparation of Sava lectin analyzed by MALDI-TOF mass spectrometry exhibits at least 4 different molecular ion [M+H]⁺ peaks, FIG. 2. This suggests 4 natural variants of the protein. Presence of other natural variants of the sequence cannot be excluded. The dominant peak has average molecular weight of 16,803 Da. The next three according to intensity are 16,769 Da, 16,617 Da and 16,583 Da. Determination of molecular weight by size exclusion chromatography (FIG. 1) and SDS-PAGE (FIG. 1), that are both low resolution techniques, showed molecular weight of ˜17 kDa.

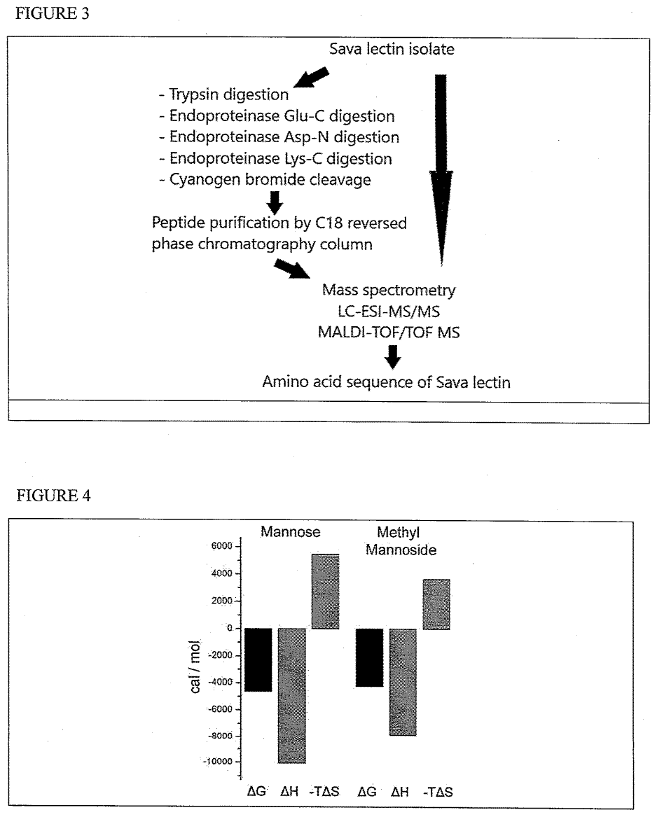

Complete amino acid sequence was determined according to the flow diagram presented at FIG. 3. Protein sample aliquots were systematically digested with different proteases and cyanogen bromide. Resulting peptide mixtures were analyzed by liquid chromatography-electrospray ionization tandem mass spectrometry (LC-ESI-MS/MS) and/or matrix assisted laser desorption ionization tandem time of flight mass spectrometry (MALDI-TOF/TOF MS). Sequence was obtained by analysis of overlapping peptides obtained by different proteases and cyanogen bromide. Neither glycosylation nor other posttranslational modifications observed.

Amino acid sequences of these four main peaks are shown in Table 1. Sequence of dominant 16,803 Da peak contains 149 amino acids. Sequence that corresponds to 16,769 Da peak is the same with one amino acid substitution Phe61Leu. Sequence of 16,617 Da is the same as sequence of 16,803 Da peak but has deletion of C terminal Trp. Sequence of 16,583 Da is the same as sequence of 16,769 Da peak but has deletion of C terminal Trp. Position 61 is underlined in each variant. Also shown is an exemplary nucleic acid sequence encoding the dominant variant. This sequence is used where recombinant Sava lectin is expressed from bacterial cells, e.g. E coli. In this context, the sequence is modified to include an additional 5′ codon for an extra N terminal methionine, to optimize expression. The optimized sequence is typically into vector pET28a+ using NcoI/XhoI restriction sites. A protein purification tag may optionally be included, but recombinant Sava lectin may also be purified by exploiting the lectin binding activity of the lectin itself (e.g. using an affinity column with carbohydrate target).

TABLE 1

| | | | | Calculated | |
|---|---|---|---|---|---|
| Average | | | | molar | |
| mol | | | Total no. | extinction | |
| weight | | Calculated | of amino | coefficient | SEQ |
| (Da) | Amino acid sequence | pI | acids | (M⁻¹ cm⁻¹) | ID |
| 16,803 | SNLRWVATSGGKVPEHAVVGGENFSGEVLYVARVRINSG | 8.96 | 149 | 41370 | 1 |
| | LTPGKMAPSYRVAHTSYGGKEFYKSDYELITNPGWKSHI | | | | |
| | EWKYHEGHSKPTNAVLGGTDQRNLYVARFMYGDGHMLSG | | | | |
| | KVLFDYGKAYLGYNNKEYESTKFYVLVEHPSW | | | | |
| 16,769 | SNLRWVATSGGKVPEHAVVGGENFSGEVLYVARVRINSG | 8.96 | 149 | 41370 | 2 |
| | LTPGKMAPSYRVAHTSYGGKELYKSDYELI TNPGWKSHI | | | | |
| | EWKYHEGHSKPTNAVLGGTDQRNLYVARFMYGDGHMLSG | | | | |
| | KVLFDYGKAYLGYNNKEYESTKFYVLVEHPSW | | | | |
| 16,617 | SNLRWVATSGGKVPEHAVVGGENFSGEVLYVARVRINSG | 8.96 | 148 | 35870 | 3 |
| | LTPGKMAPSYRVAHTSYGGKEFYKSDYELITNPGWKSHI | | | | |
| | EWKYHEGHSKPTNAVLGGTDQRNLYVARFMYGDGHMLSG | | | | |
| | KVLFDYGKAYLGYNNKEYESTKFYVLVEHPS | | | | |
| 16,583 | SNLRWVATSGGKVPEHAVVGGENFSGEVLYVARVRINSG | 8.96 | 148 | 35870 | 4 |
| | LTPGKMAPSYRVAHTSYGGKELYKSDYELITNPGWKSHI | | | | |
| | EWKYHEGHSKPTNAVLGGTDQRNLYVARFMYGDGHMLSG | | | | |
| | KVLFDYGKAYLGYNNKEYESTKFYVLVEHPS | | | | |
| | Exemplary nucleic acid sequence | | | | |
| | (encodes SEQ ID NO 1) | | | | |
| — | AGCAACCTGCGTTGGGTTGCTACTTCTGGTGGTAAAGTC | — | — | — | 5 |
| | CCTGAACACGCAGTAGTCGGTGGCGAAAACTTTTCTGGT | | | | |
| | GAAGTGCTGTACGTGGCACGTGTACGTATCAACTCTGGT | | | | |
| | CTGACGCCAGGCAAGATGGCTCCGTCTTATCGTGTTGCG | | | | |
| | CATACCTCTTACGGTGGCAAAGAATTCTACAAGAGCGAT | | | | |
| | TACGAACTGATCACTAATCCGGGCTGGAAATCCCACATT | | | | |
| | GAGTGGAAATACCACGAGGGTCACTCCAAACCGACCAAC | | | | |
| | GCGGTTCTGGGTGGTACCGATCAGCGCAACCTGTATGTA | | | | |
| | GCCCGCTTCATGTATGGTGACGGCCACATGCTGTCCGGC | | | | |
| | AAAGTTCTGTTCGACTACGGCAAAGCGTATCTGGGCTAT | | | | |
| | AACAACAAAGAATACGAATCCACCAAATTCTACGTGCTG | | | | |
| | GTTGAACATCCGAGCTGG | | | | |
| | Sequence inserted into plasmid pJL | | | | |
| | TRBO by PacI and NotI. Plasmid was | | | | |
| | inserted into Agrobacterium | | | | |
| | tumefacines (Agro GV3101). | | | | |
| | AGCAACCTCAGATGGGTTGCTACCTCGGGCGGAAAGGTG | — | — | — | 10 |
| | CCGGAGCATGCTGTTGTTGGTGGAGAGAATTTTTCTGGT | | | | |
| | GAAGTATTGTATGTGGCCCGTGTAAGGATAAATTCTGGC | | | | |
| | TTAACACCCGGCAAAATGGCTCCATCCTACCGAGTAGCT | | | | |
| | CACACTTCTTATGGGGGGAAAGAATTCTACAAGTCAGAC | | | | |
| | TATGAGCTAATCACAAATCCTGGTTGGAAATCTCATATT | | | | |
| | GAATGGAAGTATCATGAAGGTCACAGCAAACCAACTAAT | | | | |
| | GCAGTGCTGGGAGGAACTGATCAAAGGAATCTTTATGTT | | | | |
| | GCAAGATTCATGTATGGTGATGGGCATATGTTGAGTGGT | | | | |
| | AAAGTTCTTTTTGATTATGGAAAAGCATACCTTGGATAC | | | | |
| | AACAACAAGGAGTATGAATCAACGAAGTTTTATGTCTTA | | | | |
| | GTCGAACATCCTTCATGG | | | | |
| | Exemplary sequences optimized for | | | | |
| | expression = SEQ ID Nos: 1 to 4 with N | | | | |
| | terminal Met added. | | | | |
| | MSNLRWVATSGGKVPEHAVVGGENFSGEVLYVARVRINS | | | | 6 |
| | GLTPGKMAPSYRVAHTSYGGKEFYKSDYELITNPGWKSH | | | | |
| | IEWKYHEGHSKPTNAVLGGTDQRNLYVARFMYGDGHMLS | | | | |
| | GKVLFDYGKAYLGYNNKEYESTKFYVLVEHPSW | | | | |
| | MSNLRWVATSGGKVPEHAVVGGENFSGEVLYVARVRINS | | | | 7 |
| | GLTPGKMAPSYRVAHTSYGGKELYKSDYELITNPGWKSH | | | | |
| | IEWKYHEGHSKPTNAVLGGTDQRNLYVARFMYGDGHMLS | | | | |
| | GKVLFDYGKAYLGYNNKEYESTKFYVLVEHPSW | | | | |
| | MSNLRWVATSGGKVPEHAVVGGENFSGEVLYVARVRINS | | | | 8 |
| | GLTPGKMAPSYRVAHTSYGGKEFYKSDYELITNPGWKSH | | | | |
| | IEWKYHEGHSKPTNAVLGGTDQRNLYVARFMYGDGHMLS | | | | |
| | GKVLFDYGKAYLGYNNKEYESTKFYVLVEHPS | | | | |
| | MSNLRWVATSGGKVPEHAVVGGENFSGEVLYVARVRINS | | | | 9 |
| | GLTPGKMAPSYRVAHTSYGGKELYKSDYELITNPGWKSH | | | | |
| | IEWKYHEGHSKPTNAVLGGTDQRNLYVARFMYGDGHMLS | | | | |
| | GKVLFDYGKAYLGYNNKEYESTKFYVLVEHPS | | | | |

### Carbohydrate Specificity

Different techniques were used to explore carbohydrate specificity of Sava lectin.

Isothermal titration calorimetry (ITC) experiments were performed with Sava lectin at pH 7.4 in 50 mM phosphate buffered saline (PBS) at 25° C. and in the presence of different D-carbohydrates (mono- and di-saccharides).

Using ITC we did not observed binding of galactose, N-acetyl galactosamine, glucose, N-acetyl glucosamine, fucose, mannitol, rhamnose, lactose, raffinose, and arabinose. Binding of mannose and methyl mannoside was observed with ITC. Binding constants (Kb) determined by ITC for D-mannose Kb=2514 M⁻¹ and methyl alpha-D-mannoside Kb=1270 M⁻¹.

In order to explore effect of cations on carbohydrate binding activity of Sava lectin binding of carbohydrates was performed with three different approaches, 1) intensive dialysis of Sava lectin and carbohydrates, against triple distilled water, before ITC experiments, 2) using EDTA (ethylenediaminetetraacetic acid) to bind and remove free cations from solution in ITC experiments and 3) addition of inorganic salts in ITC experiments. We did not observe any changes in carbohydrate binding of Sava lectin in the presence or absence of cations, including calcium ions. Carbohydrate binding activity of at least one known class of lectins is strictly dependent on presence of calcium ions.

Turbidimetric activity assay was performed to confirm ITC results. Results were confirmed with one exception, weak binding of D-glucose was observed with binding constant two orders of magnitude lower than for D-mannose. This small binding constant produces small heat effect that cannot be observed by ITC.

Microscale thermophoresis (MST) experiments confirmed binding of mannose and methyl mannoside. Dissociation constant (Kd) for methyl alpha-D-mannoside was determined to be 13.2+/−1.74 mM. In the case of D-mannose two binding events were observed, first with Kd 26.5+/−1.47 μM and second with Kd 11.2+/−1.12 mM. Two Km values indicate existence of two binding sites that could be different of allosterically connected.

Experiments with differential scanning calorimetry confirmed binding of mannose and methyl mannoside, see discussion of thermal stability below.

### Thermodynamics of Carbohydrate Binding

Thermodynamic parameters of mannose and methyl mannoside binding were obtained by algorithm for global fitting of the model function to the experimental ITC thermograms based on the iterative non-linear Levenberg-Marquardt χ2 regression procedure. The best agreement between model and experimental data was achieved when two binding sites per protein molecule were assumed.

For both carbohydrates binding is driven by a large enthalpic contribution with unfavorable entropic contribution, see FIG. 4. A plot of ΔH versus-TAS yielded a straight line with a slope of 1.2 that indicates low enthalpy-entropy compensation.

### pH Stability

Stability of Sava lectin activity was determined using turbidimetric assay at different pH at 25° C. Different buffers were used, in the pH range 3.5-6 was acetate buffer and Tris buffer in the pH range 7-8.9.

TABLE 2
Stability of Sava lectin activity at different pH

| pH | 4 | 4.5 | 5 | 6 | 7 | 7.5 | 8 | 8.5 | 9 |
|---|---|---|---|---|---|---|---|---|---|
| Sava lectin carbohydrate | 2⁵ | 2⁵ | 2⁷ | 2⁷ | 2⁹ | 2⁸ | 2⁸ | 2⁸ | 2⁸ |
| binding activity | | | | | | | | | |

Maximal carbohydrate binding activity was observed at pH 7 (Table 2). Strong activity was observed in basic pH, while activity in acidic pH range was slightly decreased. Experiments confirm good stability of activity at different pH values. Regarding potential use of Sava lectin as topical microbicide it can be used at the skin surface and mucosal surfaces (nasal, vaginal, rectal, and oral).

### Thermal Stability

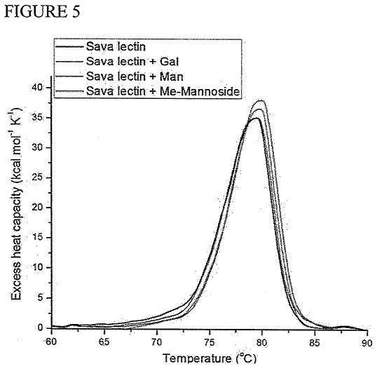

Thermal stability was determined by differential scanning calorimetry (DSC). Experiments were performed in PBS at pH 7.4. The DSC thermogram (FIG. 5) shows high thermal stability of Sava lectin with melting point (Tm) at 79.17° C. In the presence of carbohydrate that are bound to Sava lectin Tm value increase for methyl mannoside to 79.55° C. and for mannose to 79.63° C. Increased Tm indicates stabilization of protein structure in the presence of bound carbohydrate. In the presence of galactose, carbohydrate that does not bind, Tm of Sava lectin was the same as in the experiment with Sava lectin only.

Thermal denaturation of Sava lectin is kinetically controlled process with low reversibility (about 50%). Process of thermal unfolding is accompanied with precipitation phase. High thermal stability of Sava lectin is very useful characteristics necessary for broad applications including pharmacological applications.

### Influence of Carbohydrate Binding on Stability

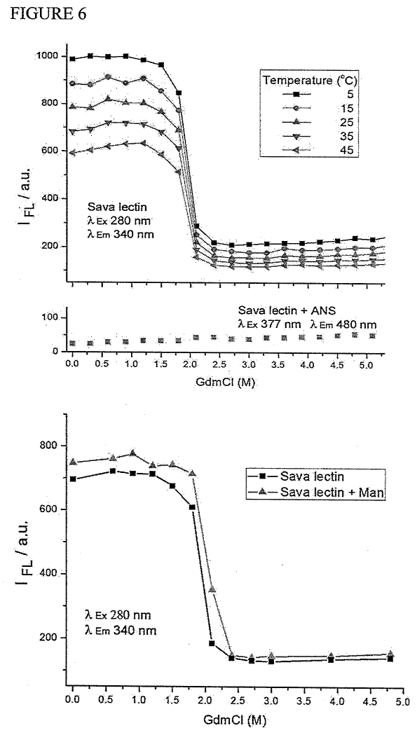

Increased stability of Sava lectin upon carbohydrate binding was confirmed by DSC, see FIG. 5. Stabilization upon carbohydrate binding was confirmed by spectrofluorimetric study of Sava lectin denaturation with guanidinium chloride (GdmCl). Denaturation with GdmCl is fully reversible and follows two state model (FIG. 6 Top). Existence of intermediates in this two state model of denaturation by GdmCl was not observed (FIG. 6 Middle). Increase in concentration of GdmCl that is necessary to induce transition between two states was observed upon binding of mannose (FIG. 6 Bottom).

### Secondary and Tertiary Structure

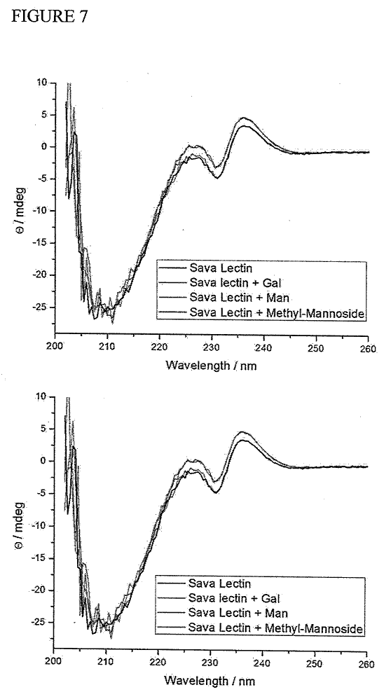

Secondary structure elements were assessed by circular dichroism (CD) spectra in Far-UV range (FIG. 7 Top). This CD spectrum of Sava lectin is characteristic for beta structured proteins. Slight changes of Far-UV CD spectrum upon binding of carbohydrates (mannose and methyl mannoside) may also be observed.

Near-UV CD spectrum (FIG. 7 Bottom) indicates existence of certain conformational changes upon binding of carbohydrates (mannose and methyl mannoside). Decrease in negative ellipticity in Near-UV CD spectra from 260 to 285 nm in the field of absorption of Trp, Tyr and Phe indicates involvement of these amino acids in ligand binding and/or some conformational changes upon ligand binding.

Tertiary structure was determined by X-ray crystallography. Crystal was obtained at pH 7.4 (FIG. 8 Top-left). Resolution of crystal structures is from 2.5 Å to 1.6 Å. Schematic representations of Sava lectin tertiary structure are shown. Based on the crystal structure, a computer modeling proposed structure of Sava lectin dimer is shown (FIG. 8 Top-right).

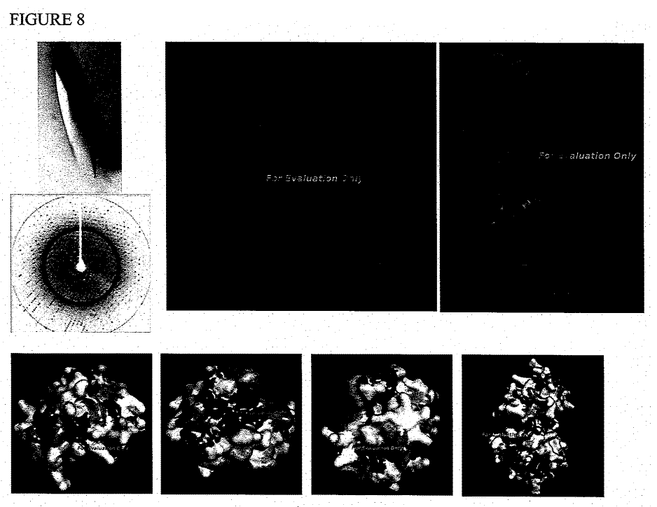

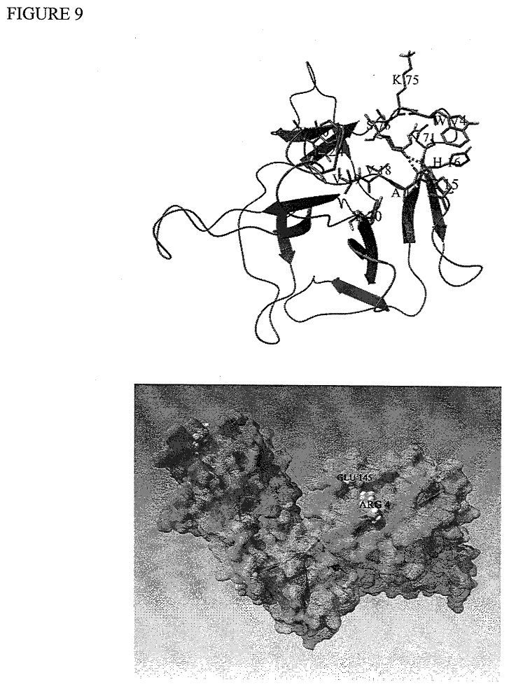

Independently from X-ray structure we have performed a computational study based on primary structure. Computer modeling proposed existence of two carbohydrate binding sites and the amino acids involved (FIG. 9 top). Also, computer modeling proposed mechanism of Sava lectin multimerization (FIG. 9 bottom).

### Quaternary Structure

Different techniques were used to explore quaternary structure of Sava lectin.

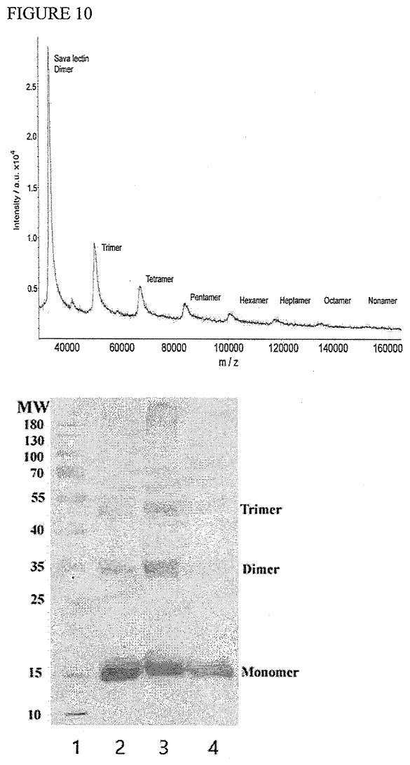

Mass spectrometric analysis of Sava lectin samples on MALDI-TOF mass spectrometer showed peaks with m/z values that correspond to multiplication of monomer Sava lectin mass (FIG. 10 Top). This result indicates existence of dimer, trimer, tetramer, pentamer, hexamer, heptamer, octamer, nonamer structures.

Another approach includes short exposure of Sava lectin solution to cross linker molecule glutaraldehyde. Upon exposure monomers that are involved in formation of oligomeric structures in solution become covalently cross linked and can be observed by denaturing electrophoresis. The SDS-PAGE analysis shows bands that correspond to multiplication of monomer Sava lectin mass (FIG. 10 Bottom).

Computer molecular modeling approach also indicated possibility of multimerisation of Sava lectin (FIG. 9 bottom).

### Cytotoxicity

Cytotoxicity of Sava lectin was analyzed against different cell lines using MTT (3-(4,5-dimethylthiazol-2-yl)-2,5-diphenyltetrazolium bromide) assay for quantification of viable cells. Results are presented in Table 3.

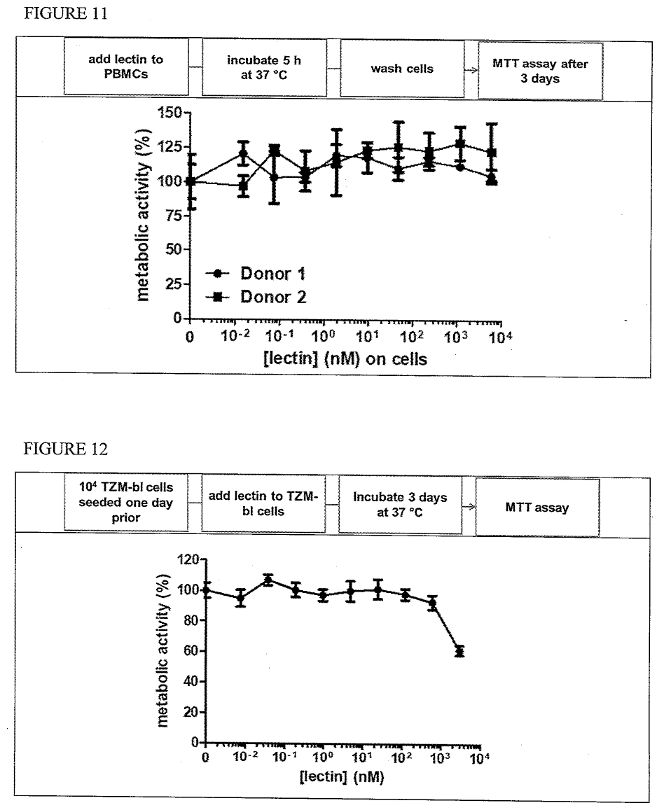

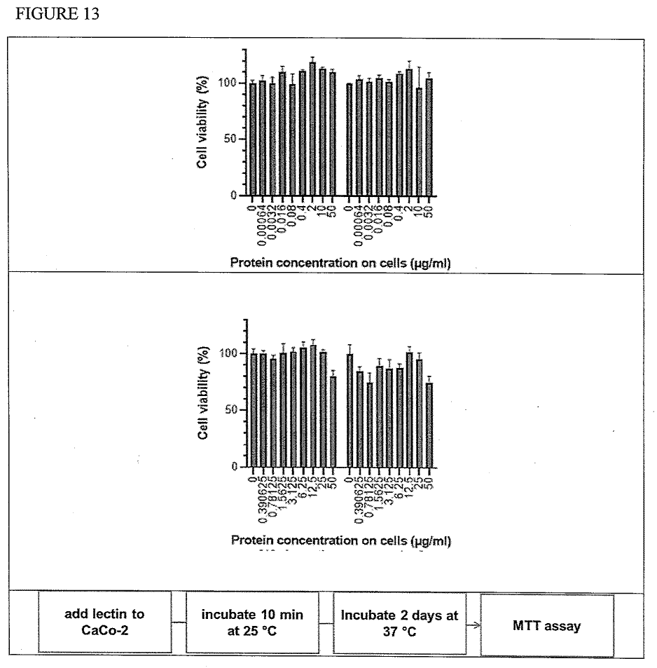

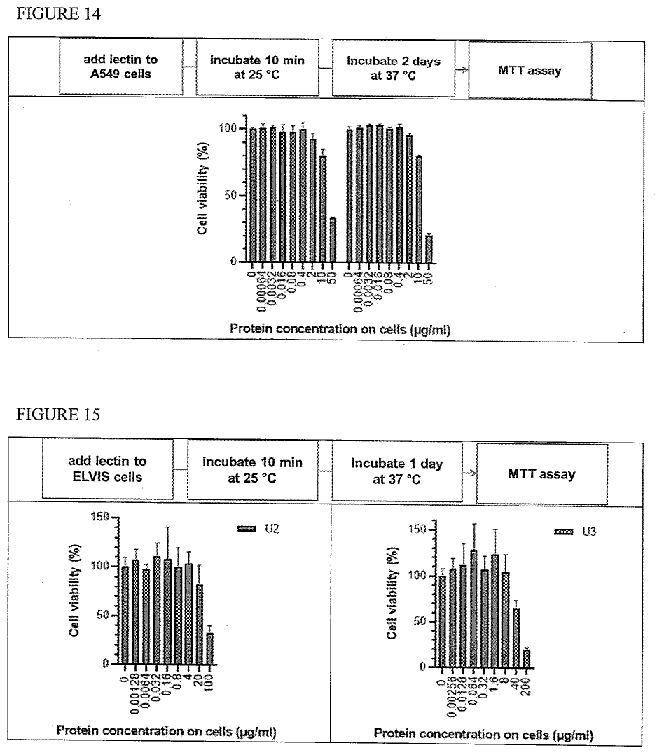

Cytotoxicity test showed low cytotoxic effect on peripheral blood mononuclear cells (PBMCs) freshly isolated from human blood FIG. 11 and Table 3. Cytotoxic concentrations are well above Sava lectin concentrations necessary for therapeutic anti-viral application Table 5. Experiments were performed separately with PBMCs of 3 different donors, with each experiment performed in triplicate.

Cytotoxic effect of Sava lectin on TZM-bl cell line (genetically altered HeLa for viral diagnostics of HIV-1) can be observed in concentrations that are much huger than concentrations necessary for anti-HIV-1 activity, FIG. 12 and Table 5.

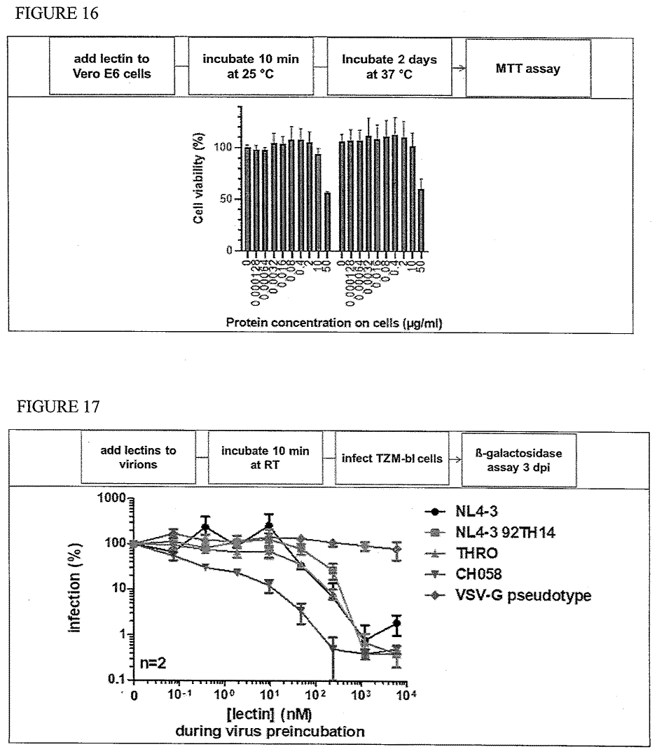

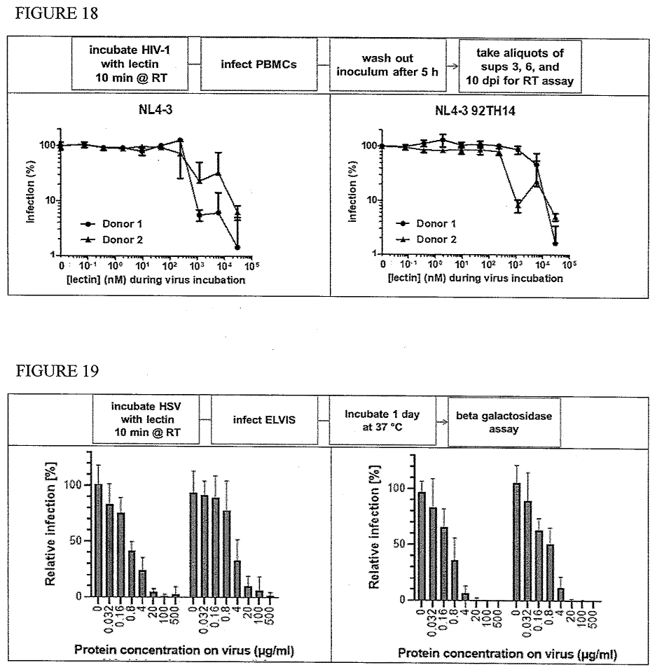

Cytotoxic effect of Sava lectin on ELVIS cell line (genetically altered baby hamster kidney cells for diagnostics of HSV-1 and HSV-2) and on Vero E6 call line (origin of cells is kidney of African green monkey) FIGS. 15 and 16 can be observed in concentrations that are much huger than concentrations necessary for anti-HSV-1 and anti-HSV-2 activity, Table 5.

Cytotoxic effect of Sava lectin on human cancer cell lines is described in the next segment-Anticancer activity.

TABLE 3
Maximal concentrations of Sava lectin that
have no effect on different cell lines

| | Maximal | | |
|---|---|---|---|
| | non-toxic | | Number of |
| | concentration | | cells per |
| Cell line | nM * | μg/ml | well |
| PBMCs | 10,000 | 168 | 10,000 |
| (human peripheral blood | 59,523 | 1000 | 100,000 |
| mononuclear cells) | | | |
| TZM-bl (genetically | 1,000 | 16.8 | 10,000 |
| altered HeLa cells) | | | |
| ELVIS (enzyme-linked virus | 476 | 8 | 5,000 |
| inducible system (ELVIS) | | | |
| cell line) (genetically altered | | | |
| baby hamster kidney cell line) | | | |
| VeroE6 (genetically altered | 595 | 10 | 6,000 |
| epithelial kidney from | | | |
| Cercopithecus aethiops) | | | |
| A549 (adenocarcinoma | 119 | 2 | 20,000 |
| human alveolar basal | | | |
| epithelial cells) | | | |
| CaCo-2 | 2,976 | 25 | 10,000 |
| (immortalized cell line of human | 1,488 | 50 | 25,000 |
| colorectal adenocarcinoma cells) | | | |
| Jurkat | 5,952 | 100 | 10,000 |
| (immortalized line of human T | | n/d | 100,000 |
| lymphocyte cells) | | | |
| U937 | 1,190 | 20 | 10,000 |
| (human myeloid leukemia cell line) | 2,976 | 50 | 100,000 |
| MDA | 1,190 | 20 | 10,000 |
| (human breast cancer cell line) | 5,952 | 100 | 100,000 |
| HeLa | 5,952 | 100 | 10,000 |
| (human cervical cancer cells) | 14,888 | 250 | 100,000 |

* Molarity was calculated taking into account molecular weight of Sava lectin monomer

### Anticancer Activity

Cytotoxic activity of Sava lectin against different human cancer cell lines was assessed using MTT test. Results are presented in Table 3 and 4. Certain cancer cell lines such as A549 show high sensitivity to Sava lectin with EC50 values in the range of several μg/ml. This result make Sava lectin candidate for a potential anticancer candidate agent, especially because Sava lectin toxicity against PBMCs is not observed in this range of concentrations.

Cancer cell lines such as CaCo-2, Jurkat and U937 are moderately sensitive to Sava lectin with EC50 values in the rage of tenths to hundreds μg/ml. Cancer cell lines such as HeLa and MDA seem to survive high concentrations of Sava lectin with EC50 values in the range several hundreds of μg/ml to mg/ml. For comparison anticancer activity test was performed also with Concanavalin A lectin (JBS 16 10, Glycobiology 22 1245). It may be seen that ConA has slightly stronger anticancer activity against certain cancer cell lines, Table 4.

Maximal non-toxic concentration that are presented in units of nM should be taken into account with the knowledge that ConA active form is tetramer, while for Sava lectin molar calculations was made taking into account molecular weight of monomer.

TABLE 4
Maximal concentrations of Sava lectin and Concanavalin A
lectin that have no effect on different cancer cell lines

| | Maximal non-toxic | | Maximal non-toxic | | Number |
|---|---|---|---|---|---|
| | conc of Sava lectin | | conc of ConA | | of cells |
| Cell line | nM * | μg/ml | nM | μg/ml | per well |
| A549 (adenocarcinoma human | 119 | 2 | | n/d | 20,000 |
| alveolar basal epithelial cells) | | | | | |
| CaCo-2 | 2,976 | 25 | | n/d | 10,000 |
| (immortalized cell line of human | 1,488 | 50 | | n/d | 25,000 |
| colorectal adenocarcinoma cells) | | | | | |
| Jurkat | 5,952 | 100 | 926 | 100 | 10,000 |
| (immortalized line of human T | | n/d | 926 | 100 | 100,000 |
| lymphocyte cells) | | | | | |
| U937 | 1,190 | 20 | 926 | 100 | 10,000 |
| (human myeloid leukemia cell line) | 2,976 | 50 | 463 | 50 | 100,000 |
| MDA | 1,190 | 20 | 185 | 20 | 10,000 |
| (breast cancer cell line) | 5,952 | 100 | 463 | 50 | 100,000 |
| HeLa | 5,952 | 100 | 463 | 50 | 10,000 |
| (cervical cancer cells) | 14,888 | 250 | 463 | 50 | 100,000 |

*Molarity was calculated taking into account molecular weight of Sava lectin monomer

### Mitogenic Activity

It is well known that lectins may induce proliferation of cells. This property is one of the main obstacles for application of lectins in medicine as microbicide agents. Mitogenic effect (induction of mitosis) of certain compound is characterized by increased DNA synthesis. A Bromodeoxyuridine (BrdU) incorporation assay was applied to detect DNA synthesis. Mitogenic effect of Sava lectin was examined using BrdU assay and peripheral blood mononuclear cells (PBMCs). Mitogenic effect on PBMCs was not observed.

### Antiviral Activity

Potential antiviral activity of Sava lectin was examined by different enveloped viruses and virus strains in different cells, Table 5. Sava lectin exhibits concentration dependent inhibition of virus induced decrease of cell viability. This inhibition is not equal far all enveloped viruses and their strains. The most potent inhibition was observed for CH058 strain of HIV-1 with inhibitory concentration in the picomolar range. The range for EC50 inhibitory concentrations for HIV-1 strains used in this study is 0.05-60 nM, FIG. 17 and Table 5. This result is in the same range (0.03-56 nM) with anti HIV-1 activity of the most potent published lectin griffithsin (JBC 280 9345). The VSV-G (the vesicular stomatitis virus glycoprotein) pseudotype HIV-1 Δenv are not identical to the genuine HIV-1 particles and does not engage CD4 and CCR5 or CXCR4 receptors for fusion and entry into the cell (PLOS Pathog 5 1000633). Therefore, absence of antiviral activity of Sava lectin against this pseudotype confirms that antiviral activity of Sava lectin is through inhibition of binding of HIV-1 gp120 to receptors.

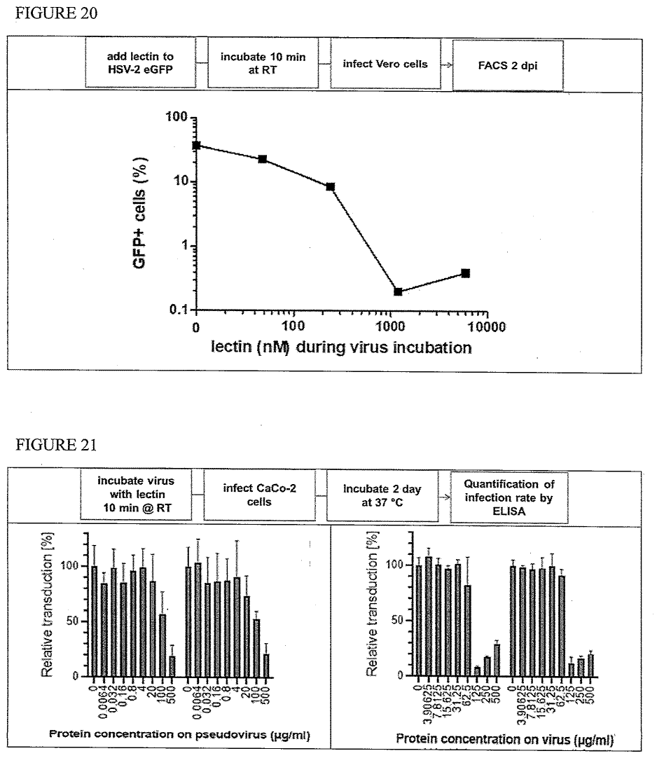

Potent antiviral activity in low nanomolar range was confirmed for Herpes simplex virus, see FIGS. 19 and 20 and Table 5. Antiviral activity in micromolar range was observed for severe acute respiratory syndrome coronavirus 2, FIG. 21 and Table 5.

Strong binding of Sava lectin to Influenza A virus wild type (Washington), H1N1 (Guangdong-Maonan) and H3N2 (Hong Kong) (data not shown) was confirmed and antiviral activity can be expected. Binding to human HCMV, Zika virus, Measles virus were observed but antiviral activity required very high concentrations of Sava lectin i.e. weak antiviral activity was observed. In a comparison of binding, Sava lectin shows stronger binding to Influenza A than Banana lectin (data not shown) which suggests Sava lectin will have good antiviral properties against Influenza A. Banana lectin (version H84T) is potent anti-Influenza agent.

There are many other viruses that engage glycoproteins from viral envelope in fusion and entry into the cell and that, based on these results and known properties of the viruses, can be expected to be inhibited by Sava lectin. Thus, in addition to the direct demonstrations above, it is expected that Sava lectin will have antiviral effects for at least Hepatitis C (HCV) and B virus (HBV), Influenza A and B virus strains, SARS-COV-1, Ebola virus, Dengue virus, West Nile virus, Yellow fever virus, Japanese encephalitis virus, Porcine epidemic diarrhea virus, Simian immunodeficiency virus.

### Anti-Bacterial and Anti-Yeast Activity

Potential antibacterial activity of Sava lectin was explored against different bacterial species: S. aureus (ATCC 25923), P. aeruginosa (ATCC 27853), E. coli (ATCC 25922), B. cereus (ATCC 11778), E. faecalis (ATCC 29212) and S. typhimurium (ATCC 14028). Standard test for determination of minimal inhibitory concentrations (MIC) did not show inhibition in the range of concentrations 0.0625-1 mg/ml. Small inhibition of growth was observed in the E. coli. However, this test is performed in Luria Bertani medium that contains extract of S. cerveisiae that is rich in high mannose glycans that strongly binds Sava lectin and consequently inhibit its activity. When a test of contact inhibition was performed in PBS, which includes 20 min exposure of 5×10⁵ S. aureus (ATCC 25923) cells to 0.18 mg/ml Sava lectin, showed more than 25% inhibition of growth. This result confirms that interaction between Sava lectin and bacteria can be potentially used to inhibit growth of bacteria. This might help in future efforts to develop safer and more effective microbial control agents. Binding of Sava lectin to several different species of bacterial genus Lactobacilli was explored. Significant binding was not observed.

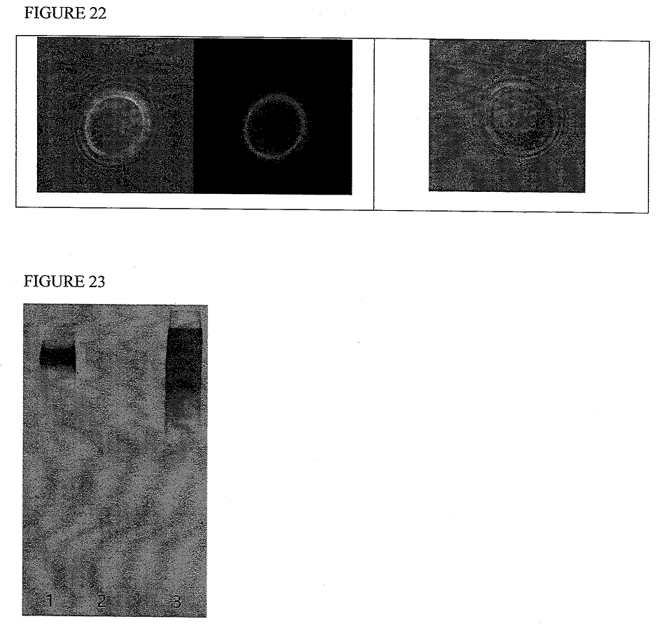

Potential activity of Sava lectin against yeasts was explored against three species: C. glabrata (h65b C.g), C. albicans (ATCC 10231) and Saccharomyces cerevisiae (ATCC 9763). Standard test for determination of minimal inhibitory concentrations (MIC) did not show inhibition in the range of concentrations 0.03125 to 1 mg/ml. As in the case of MIC test for bacteria, components of media used for yeast growth (Tryptic Soy Broth and Pepton 10) contain glycan structures that inhibit activity of Sava lectin. The test of contact inhibition performed in PBS, with 20 min exposure of 2×10³ S. cerevisiae (ATCC 9763) cells to 0.18 mg/ml Sava lectin, showed about 35% inhibition of growth. Binding of Sava lectin to the cell wall mannans of S. cerevisiae and C. albicans was confirmed by FITC labeled Sava lectin, see FIG. 22.

### Application in Life Science and Biotechnology

Usually classification of lectins is according to carbohydrate specificity. However, lectins from each class, for example mannose binding lectins, have fine differences in carbohydrate binding specificities. These differences are observed by carbohydrate micro array. One such carbohydrate (glycan) microarray was developed by Consortium for Functional glycomis. Having in mind this information every new lectin open possibilities for development of specific products.

For example development of lectin-linked immunomagnetic separation for the detection of hepatitis a virus was described by Sung-Mu at all. (Viruses 6 1037).

We have used FITC labeled Sava lectin to explore binding to S. cerevisiae and C. albicans by confocal microscopy and ELISA assay. Results of both assays confirmed binding of Sava lectin to cell wall of these two yeasts. Confocal microscopy confirms that Sava lectin cannot enter yeast cell and remains bound to the cell surface that is observed as bright (green) ring around the cell, see FIG. 22.

Sava lectin can be used for detection of glycoproteins. For this application we have labeled Sava lectin with biotin. The avidin-biotin is usual biochemical system for coupling of proteins used in many biological assays. Results of western blot detection of glycoproteins with biotinylated Sava lectin and subsequent coupling of streptavidin-HRP for visualization by DAB, are presented at FIG. 23. This result confirms that Sava lectin can be used in all different systems for affinity labeling, binding, capture.

### Recombinant Production

Recombinant production of Sava lectin was achieved in two standard expression systems Escherichia coli and Nicotiana benthamiana. Genes were synthesized according to amino acid sequence and his-tag was added for expression in E. coli. Synthetic genes were incorporated into expression plasmid suitable for expression system.

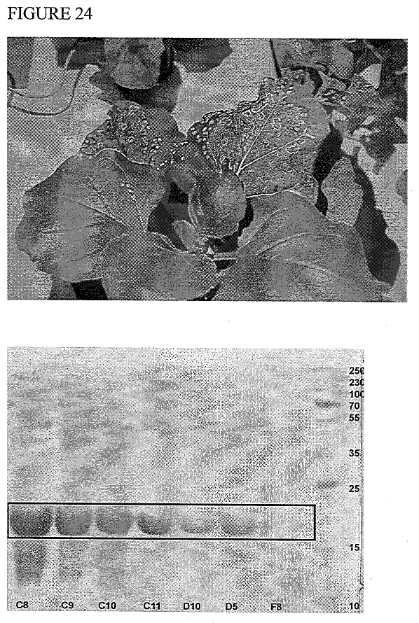

In the case of E. coli purified expression plasmids (pET28 and pMSCG7) were introduced separately into competent BL21 E. coli cells. Both fermentations ware performed for 10 h at 37° C. and expressed protein was purified from harvested cells. Yield of Sava lectin was good, see FIG. 24 Bottom. However, amount of properly folded protein with carbohydrate binding activity was small.

In the case of N. benthamiana expression vector (pJL-TRBO) was introduced by electroporation into competent laboratory strain Agrobacterium tumefaciens GV3101. Successfully transformed A. tumefaciens cells were selected and with colony PCR confirmed. These A. tumefaciens cells were introduced into 5 weeks old leaves of N. benthamiana by agroinfiltration. Successful expression was confirmed by co-expression of green fluorescent protein, see FIG. 24. Leaves were harvested after 5 days and expressed protein was purified from leaves. Yield of properly folded protein was much better than in the case of E. coli.

Table of literature references

| Short identifier | |
|---|---|
| used in this | |
| document | Detailed description |
| AAC 54 1425 | Jan Balzarini, Katrien O. Francois, Kristel Van Laethem, Bart Hoorelbeke, Marleen Renders, |
| | Joeri Auwerx, Sandra Liekens, Toshikazu Oki, Yasuhiro Igarashi, Dominique Schols. Pradimicin |
| | S, a Highly Soluble Nonpeptidic Small-Size Carbohydrate-Binding Antibiotic, Is an Anti-HIV |
| | Drug Lead for both Microbicidal and Systemic Use. Antimicrobial agents and chemotherapy 54 |
| | (2010) 1425-1435 doi: 10.1128/AAC.01347-09 |
| AMB 86 805 | Sheng Xiong, Jun Fan, Kaio Kitazato. The antiviral protein Cyanovirin-N: The current state of its |
| | production and applications. Applied Microbiology and Biotechnology volume 86 (2010) 805- |
| | 812. doi: 10.1007/s00253-010-2470-1 |
| AR 18 191 | Jan Balzarini, Johan Neyts, Dominique Schols, Mitsuaki Hosoya, Els Van Damme, Willy |
| | Peumans, Erik De Clercq. The mannose-specific plant lectins from Cymbidium hybrid and |
| | Epipactis helleborine and the (N-acetylglucosamine)n-specific plant lectin from Urtica dioica are |
| | potent and selective inhibitors of human immunodeficiency virus and cytomegalovirus replication |
| | in vitro. Antiviral Research, 18 (1992) 191-207. |
| AR 71 237 | Jan Balzarini. Inhibition of HIV entry by carbohydrate-binding proteins. Antiviral Research 71 |
| | (2006) 237-247. |
| ARHR 28 1513 | Geoffrey Ferir, Dana Huskens, Kenneth E. Palmer, Daniel M. Boudreaux, Michael D. Swanson, |
| | David M. Markovitz, Jan Balzarini, Dominique Schols. Combinations of Griffithsin with Other |
| | Carbohydrate-Binding Agents Demonstrate Superior Activity Against HIV Type 1, HIV Type 2, |
| | and Selected Carbohydrate-Binding Agent-Resistant HIV Type 1 Strains. Aids research and |
| | human retroviruses 28 (2012) 1513-1523 doi: 10.1089/aid.2012.0026 |
| BBA 1572 165 | Hans-Joachim Gabius, Sabine Andre, Herbert Kaltner, Hans-Christian Siebert. The sugar code: |
| | functional lectinomics. Biochimica et Biophysica Acta General subjects 1572 (2002) 165-177. |
| BBA 1572 198 | Remy Loris. Principles of structures of animal and plant lectins. Biochimica et Biophysica Acta |
| | General subjects 1572 (2002) 198-208. |
| COV 0 95 | Leonardus M. I. Koharudin, Angela M. Gronenborn. Antiviral lectins as potential HIV |
| | microbicides. Curr Opin Virol. 0 (2014) 95-100. doi: 10.1016/j.coviro.2014.05.006 |
| EJB 169 97 | Zoran KLJAJIC, Heinz C. SCHRODER, Michael ROTTMANN, Margita CUPERLOVIC, |
| | Miodrag MOVSESIAN, Gerhard UHLENBRUCK, Miroslav GASIC, Rudolf K. ZAHN, Werner |
| | E. G. MULLER. A D-mannose-specific lectin from Gerardia savaglia that inhibits |
| | nucleocytoplasmic transport of mRNA. European Journal of Biochemistry 169 (1987) 97-104. |
| Glycobiology 22 | Pratt J, Roy R, Annabi B. Concanavalin-A-induced autophagy biomarkers requires membrane |
| 1245 | type-1 matrix metalloproteinase intracellular signaling in glioblastoma cells. Glycobiology 22 |
| | (2012) 1245-1255. |
| JAC 69 582 | Leen Mathys, Jan Balzarini. Exposure of HIV-1 to a combination of two carbohydrate-binding |
| | agents markedly delays drug resistance development and selects for virus strains with |
| | compromised fitness. J Antimicrob Chemother 69 (2014) 582-593 doi: 10.1093/jac/dkt414 |
| JAIDS 1 453 | W E Müller, K Renneisen, M H Kreuter, H C Schröder, I Winkler. The D-mannose-specific lectin |
| | from Gerardia savaglia blocks binding of human immunodeficiency virus type I to H9 cells and |
| | human lymphocytes in vitro. J Acquir Immune Defic Syndr 1 (1988) 453-458. |
| JBC 280 9345 | Toshiyuki Mori, Barry R. O'Keefe, Raymond C. Sowder II, Scott Bringans, Roberta Gardella, |
| | Shannon Berg, Pamela Cochran, Jim A. Turpin, Robert W. Buckheit, Jr., James B. McMahon, |
| | Michael R. BoydaIsolation and Characterization of Griffithsin, a Novel HIV-inactivating Protein, |
| | from the Red Alga Griffithsia sp. The journal of biological chemistry 280 (2005) 9345-9353. |
| JBC 280 41005 | Jan Balzarini, Kristel Van Laethem, Sigrid Hatse, Matheus Froeyen, Willy Peumans, Els Van |
| | Damme, Dominique Schols. Carbohydrate-binding Agents Cause Deletions of Highly Conserved |
| | Glycosylation Sites in HIV GP120. J. Biol. Chem. 280 (2005) 41005-41014. doi: |
| | 10.1074/jbc.M508801200 |
| JBC 286 42900 | Katrien O. François, Jan Balzarini. The Highly Conserved Glycan at |
| | Asparagine 260 of HIV-1 gp120 Is Indispensable for Viral Entry. The |
| | journal of biological chemistry 286 (2011) 42900-42910. |
| JBS 16 10 | Lei HY, Chang CP. Lectin of Concanavalin A as an antihepatoma therapeutic agent. J. Biomed. |
| | Sci. 16 (2009) 10. |
| JV 80 8411 | Jan Balzarini, Kristel Van Laethem, Willy J. Peumans, Els J. M. Van Damme, Anders Bolmstedt, |
| | Federico Gago, Dominique Schols. Mutational Pathways, Resistance Profile, and Side Effects of |
| | Cyanovirin Relative to Human Immunodeficiency Virus Type 1 Strains with N-Glycan Deletions |
| | in Their gp120 Envelopes. Journal of virology 80 (2006) 8411-8421. doi: 10.1128/JVI.00369-06 |
| Lancet 369 787 | Jan Balzarini, Lut Van Damme. Microbicide drug candidates to prevent HIV infection. The |
| | Lancet 369 (2007) 787-797. |
| MP 74 330 | Katrien O. Francois, Joeri Auwerx, Dominique Schols, Jan Balzarini. Simian Immunodeficiency |
| | Virus Is Susceptible to Inhibition by Carbohydrate-Binding Agents in a Manner Similar to That |
| | of HIV: Implications for Further Preclinical Drug Development. Molecular Pharmacology 74 |
| | (2008) 330-337 |
| NR Microbiology | Jan Balzarini. Targeting the glycans of glycoproteins: a novel paradigm for antiviral therapy. |
| 5 583 *** | Nature Reviews Microbiology 5 (2007) 583-597 doi: 10.1038/nrmicro1707 |
| NRDD 14 111 | Alan L. Harvey, RuAngelie Edrada-Ebel, Ronald J. Quinn. The re-emergence of natural products |
| | for drug discovery in the genomics era. Nature Reviews Drug Discovery 14 (2015) 111-129. doi: |
| | 10.1038/nrd4510 |
| NRDD 20 200 | Atanas G. Atanasov, Sergey B. Zotchev, Verena M. Dirsch, the International |
| | Natural Product Sciences Taskforce, Claudiu T. Supuran. Natural products in drug discovery: |
| | advances and opportunities. Nature Reviews Drug Discovery 20 (2021) 200-216. |
| PLOS One 5 | Ewa D. Micewicz, Amy L. Cole, Chun-Ling Jung, Hai Luong, Martin L. Phillips, Pratikhya |
| 14360 | Pratikhya, Shantanu Sharma, Alan J. Waring, Alexander M. Cole, Piotr Ruchala. Grifonin-1: A |
| | Small HIV-1 Entry Inhibitor Derived from the Algal Lectin, Griffithsin. PloS ONE 5 (2010) |
| | e14360. doi: 10.1371/journal.pone.0014360 |
| PLoS One | Bonomelli C, Doores K J, Dunlop D C et al. The glycan shield of HIV is predominantly |
| 23521 | oligomannose independently of production system or viral clade. PloS One 6 (2011) e23521 |
| PLoS Pathog 5 | Dongyang Yu, Weifeng Wang, Alyson Yoder, Mark Spear, Yuntao Wu. The HIV Envelope but Not |
| 1000633 | VSV Glycoprotein Is Capable of Mediating HIV Latent Infection of Resting CD4 T Cells. PLoS |
| | Pathogens 5 (2009) e1000633. doi: 10.1371/journal.ppat.1000633 |
| SR 9 18120 | Günaydin, G., Edfeldt, G., Garber, D. A. et al. Impact of Q-Griffithsin anti-HIV microbicide gel in |
| | non-human primates: In situ analyses of epithelial and immune cell markers in rectal mucosa. Sci |
| | Rep 9 (2019) 18120. doi: 10.1038/s41598-019-54493-4 |
| Structure 14 | Natasza E. Ziolkowska, Barry R. O'Keefe, Toshiyuki Mori, Charles Zhu, Barbara Giomarelli, |
| 1127 | Fakhrieh Vojdani, Kenneth E. Palmer, James B. McMahon, Alexander Wlodawer. Domain- |
| | Swapped Structure of the Potent Antiviral Protein Griffithsin and Its Mode of Carbohydrate |
| | Binding. Structure 14 (2006) 1127-1135 doi: 10.1016/j.str.2006.05.017 |
| US | METHOD FOR REMOVAL OF VIRUSES FROM BLOOD BY LECTIN |
| 2004/0175291 | AFFINITY HEMODIALYSIS. US 2004/0175291 A1 |
| U.S. Pat. No. | METHOD FOR REMOVAL OF VIRUSES FROM BLOOD BY LECTIN |
| 7,226,429 | AFFINITY HEMODIALYSIS. Appl. No.: 10/760,810 U.S. Pat. No. 7,226,429 B2 |
| US | GRIFFITHSIN, GLYCOSYLATION-RESISTANT GRIFFITHSIN, AND RELATED |
| 2009/0092557 | CONJUGATES, COMPOSITIONS, NUCLEIC ACIDS, VECTORS, HOST CELLS, |
| | METHODS OF PRODUCTION AND METHODS OF USE. PCT/US05/18778 |
| | US 2009/0092557 A1 |
| US | GRIFFITHSIN, GLYCOSYLATION-RESISTANT GRIFFITHSIN, AND RELATED |
| 2011/0189105 | CONJUGATES, COMPOSITIONS, NUCLEIC ACIDS, VECTORS, HOST CELLS, |
| | METHODS OF PRODUCTION AND METHODS OF USE. Appl′ N05 |
| | 13/022,289 US 2011/0189105 A1 |
| U.S. Pat. No. | ANTI-VIRAL GRIFFITHSIN COMPOUNDS, COMPOSITIONS |
| 8,088,729 | AND METHODS OF USE. PCT No.: PCT/US2006/045930 |
| | U.S. Pat. No. 8,088,729 B2 |
| Virology 402 | Kabamba B. Alexandre, Elin S. Gray, Bronwen E. Lambson, Penny L. Moore, Isaac A. Choge, |
| 187 | Koleka Mlisana, Salim S. Abdool Karim, James McMahon, Barry O'Keefe, Rachel Chikwamba, |
| | Lynn Morris. Mannose-rich glycosylation patterns on HIV-1 subtype C gp120 and sensitivity to |
| | the lectins, Griffithsin, Cyanovirin-N and Scytovirin. Virology 402 (2010) 187-196. |
| | doi: 10.1016/j.virol.2010.03.021 |
| Viruses 6 1037 | Sang-Mu Ko, Joseph Kwon, Bipin Vaidya, Jong Soon Choi, Hee-Min Lee, Myung-Joo Oh, |
| | Hyeun-Jong Bae, Se-Young Cho, Kyung-Seo Oh, Duwoon Kim. Development of lectin-linked |
| | immunomagnetic separation for the detection of hepatitis a virus. Viruses 6 (2014) 1037-1048. |
| | doi: 10.3390/v6031037 |

1. A polypeptide, optionally an engineered polypeptide, having specific carbohydrate-binding activity, wherein said polypeptide comprises, consists essentially of, or consists of:
(a) the amino acid sequence of any one of SEQ ID NOs: 1, 2, 3 or 4;
(b) an amino acid sequence which is a fragment of the sequence of any one of SEQ ID NOs: 1, 2, 3 or 4, which fragment comprises at least 30 contiguous amino acids of the said sequence;
(c) an amino acid sequence which is at least 50% identical to the amino acid sequence of any one of (a), (b) or (c).

2. The polypeptide according to claim 1, which is engineered to include an additional methionine at the N terminus and/or a protein purification or other tag at the C terminus, which tag may be joined to the C terminus by a linker.

3. The polypeptide according to claim 1 which comprises or consists of the amino acid sequence of SEQ ID NO: 6, 7, 8 or 9.

4. The polypeptide according to claim 1 which has specific binding activity for glycans containing mannose.

5. The polypeptide according to claim 1 which is conjugated to an additional moiety.

6. The polypeptide according to claim 1 which has anti-pathogen activity against a pathogen which expresses a carbohydrate specifically bound by the polypeptide, optionally wherein the pathogen is a virus, a bacterial cell, a fungal cell, or a cancer cell.

7. A polynucleotide or expression vector which comprises a nucleic acid sequence encoding a polypeptide of claim 1.

8. A host cell comprising the polynucleotide or expression vector of claim 6, which is preferably bacterial cell or a plant cell, optionally wherein the bacterial cell is a cell of E. coli, Lactobacillus, or preferably A. tumefaciens, and/or wherein the plant cell is a cell of Nicotiana benthamiana.

9. The polypeptide according to claim 1, wherein the polypeptide is provided in solution, lyophilised, or immobilised, optionally together with an effective amount of at least one preservative.

10. A composition comprising a polypeptide according to claim 1 together with at least one preservative, and optionally one or more carriers, excipients, diluents or vehicles; optionally wherein the composition is pharmaceutically acceptable or is suitable for use as a surface microbicide.

11. A method comprising administering a polypeptide according to claim 1 to a sample or a subject in which carbohydrate is present.

12. The method of claim 11, which is for the ex vivo binding of carbohydrate in a sample, and which comprises administering said polypeptide to the sample and incubating under conditions suitable for binding to occur.

13. The method of claim 12 which additionally comprises the separation, detection or analysis of the resulting bound products.

14. The method of claim 11, which is for the prevention or treatment of a disease or condition in a subject, and which method comprises administering said polypeptide to the subject in a prophylactically or therapeutically effective amount.

15. The method of claim 14, wherein said disease or condition is a disease or condition mediated in whole or in part by a pathogen which expresses a carbohydrate specifically bound by the polypeptide, optionally wherein the carbohydrate is present as a glycan component of a glycoprotein or other structure present on the pathogen surface.

16. The method of claim 14, wherein the pathogen is a virus, a bacterial cell, a fungal cell, or a cancer cell.


SEQUENCE LISTING (10 sequences)

SEQ ID NO: 1 - protein, 149 aa, Savalia (Gerardia) savaglia
SNLRWVATSGGKVPEHAVVGGENFSGEVLYVARVRINSGLTPGKMAPSYRVAHTSYGGKEFYKSDYELITNPGWKSHIEWKYHEGHSKPTNAVLGGTDQRNLYVARFMYGDGHMLSGKVLFDYGKAYLGYNNKEYESTKFYVLVEHPSW

SEQ ID NO: 2 - protein, 149 aa, Savalia (Gerardia) savaglia
SNLRWVATSGGKVPEHAVVGGENFSGEVLYVARVRINSGLTPGKMAPSYRVAHTSYGGKELYKSDYELITNPGWKSHIEWKYHEGHSKPTNAVLGGTDQRNLYVARFMYGDGHMLSGKVLFDYGKAYLGYNNKEYESTKFYVLVEHPSW

SEQ ID NO: 3 - protein, 148 aa, Savalia (Gerardia) savaglia
SNLRWVATSGGKVPEHAVVGGENFSGEVLYVARVRINSGLTPGKMAPSYRVAHTSYGGKEFYKSDYELITNPGWKSHIEWKYHEGHSKPTNAVLGGTDQRNLYVARFMYGDGHMLSGKVLFDYGKAYLGYNNKEYESTKFYVLVEHPS

SEQ ID NO: 4 - protein, 148 aa, Savalia (Gerardia) savaglia
SNLRWVATSGGKVPEHAVVGGENFSGEVLYVARVRINSGLTPGKMAPSYRVAHTSYGGKELYKSDYELITNPGWKSHIEWKYHEGHSKPTNAVLGGTDQRNLYVARFMYGDGHMLSGKVLFDYGKAYLGYNNKEYESTKFYVLVEHPS

SEQ ID NO: 5 - DNA, 447 bp, Artificial Sequence
  polynucleotide encoding Sava lectin
agcaacctgcgttgggttgctacttctggtggtaaagtccctgaacacgcagtagtcggtggcgaaaacttttctggtgaagtgctgtacgtggcacgtgtacgtatcaactctggtctgacgccaggcaagatggctccgtcttatcgtgttgcgcatacctcttacggtggcaaagaattctacaagagcgattacgaactgatcactaatccgggctggaaatcccacattgagtggaaataccacgagggtcactccaaaccgaccaacgcggttctgggtggtaccgatcagcgcaacctgtatgtagcccgcttcatgtatggtgacggccacatgctgtccggcaaagttctgttcgactacggcaaagcgtatctgggctataacaacaaagaatacgaatccaccaaattctacgtgctggttgaacatccgagctgg

SEQ ID NO: 6 - protein, 150 aa, Artificial sequence
  polypeptide of SEQ ID NO 1 with additional N terminal Met
MSNLRWVATSGGKVPEHAVVGGENFSGEVLYVARVRINSGLTPGKMAPSYRVAHTSYGGKEFYKSDYELITNPGWKSHIEWKYHEGHSKPTNAVLGGTDQRNLYVARFMYGDGHMLSGKVLFDYGKAYLGYNNKEYESTKFYVLVEHPSW

SEQ ID NO: 7 - protein, 150 aa, Artificial Sequence
  polypeptide of SEQ ID NO 2 with additional N terminal Met
MSNLRWVATSGGKVPEHAVVGGENFSGEVLYVARVRINSGLTPGKMAPSYRVAHTSYGGKELYKSDYELITNPGWKSHIEWKYHEGHSKPTNAVLGGTDQRNLYVARFMYGDGHMLSGKVLFDYGKAYLGYNNKEYESTKFYVLVEHPSW

SEQ ID NO: 8 - protein, 149 aa, Artificial sequence
  polypeptide of SEQ ID NO 3 with additional N terminal Met
MSNLRWVATSGGKVPEHAVVGGENFSGEVLYVARVRINSGLTPGKMAPSYRVAHTSYGGKEFYKSDYELITNPGWKSHIEWKYHEGHSKPTNAVLGGTDQRNLYVARFMYGDGHMLSGKVLFDYGKAYLGYNNKEYESTKFYVLVEHPS

SEQ ID NO: 9 - protein, 149 aa, Artificial Sequence
  polypeptide of SEQ ID NO 4 with additional N terminal Met
MSNLRWVATSGGKVPEHAVVGGENFSGEVLYVARVRINSGLTPGKMAPSYRVAHTSYGGKELYKSDYELITNPGWKSHIEWKYHEGHSKPTNAVLGGTDQRNLYVARFMYGDGHMLSGKVLFDYGKAYLGYNNKEYESTKFYVLVEHPS

SEQ ID NO: 10 - DNA, 447 bp, Artificial Sequence
  polynucleotide encoding Sava lectin
agcaacctcagatgggttgctacctcgggcggaaaggtgccggagcatgctgttgttggtggagagaatttttctggtgaagtattgtatgtggcccgtgtaaggataaattctggcttaacacccggcaaaatggctccatcctaccgagtagctcacacttcttatggggggaaagaattctacaagtcagactatgagctaatcacaaatcctggttggaaatctcatattgaatggaagtatcatgaaggtcacagcaaaccaactaatgcagtgctgggaggaactgatcaaaggaatctttatgttgcaagattcatgtatggtgatgggcatatgttgagtggtaaagttctttttgattatggaaaagcataccttggatacaacaacaaggagtatgaatcaacgaagttttatgtcttagtcgaacatccttcatgg